SEQUENCE LISTING (34 sequences)

SEQ ID NO: 1 - protein, 14 aa, Artificial Sequence
  Sequence is synthesized
KSLSLSLSLSLSLK

SEQ ID NO: 2 - protein, 23 aa, Artificial Sequence
  SBP1 Domain
IEEQAKTFLDKFNHEAEDLFYQS

SEQ ID NO: 3 - protein, 23 aa, Artificial Sequence
  SBP1sc Domain
YIQESEFQLADKETAFELHDNKF

SEQ ID NO: 4 - protein, 16 aa, Artificial Sequence
  Self-Assembling Peptide
KKSLSLSLSLSLSLKK

SEQ ID NO: 5 - protein, 16 aa, Artificial Sequence
  Self-Assembling Peptide
EESLSLSLSLSLSLEE

SEQ ID NO: 6 - protein, 16 aa, Artificial Sequence
  Self-Assembling Peptide
RRSLSLSLSLSLSLRR

SEQ ID NO: 7 - protein, 16 aa, Artificial Sequence
  Self-Assembling Peptide(SAP)
DDSLSLSLSLSLSLDD

SEQ ID NO: 8 - protein, 40 aa, Artificial Sequence
  SAP-SBP1 Conjugates
KKSLSLSLSLSLSLKKGIEEQAKTFLDKFNHEAEDLFYQS

SEQ ID NO: 9 - protein, 40 aa, Artificial Sequence
  SAP-SBP1 Conjugates
EESLSLSLSLSLSLEEGIEEQAKTFLDKFNHEAEDLFYQS

SEQ ID NO: 10 - protein, 40 aa, Artificial Sequence
  SAP-SBP1 Conjugates
RRSLSLSLSLSLSLRRGIEEQAKTFLDKFNHEAEDLFYQS

SEQ ID NO: 11 - protein, 40 aa, Artificial Sequence
  SAP-SBP1 Conjugates
DDSLSLSLSLSLSLDDGIEEQAKTFLDKFNHEAEDLFYQS

SEQ ID NO: 12 - protein, 15 aa, Artificial Sequence
  Mimic Base-Fibrillizing Domain with glycol spacer
KSLSLSLSLSLSLKG

SEQ ID NO: 13 - protein, 84 aa, Artificial Sequence
  ACE2 receptors
EEVIAWNSNNLDSKVGGNYNYLYRCFRKSNLKPFERDISTEIYQAGSTPGNGVEGFNGYFCLQSYGFQPTNGVGYQPYRVVRRR

SEQ ID NO: 14 - protein, 78 aa, Artificial Sequence
  ACE2 receptors
EEVIAWNSNNDSKVGGNYNYLYRCKMSECVLGQSKRVQALLFNKVTLAGFNGYFCLQSYGFQPTNGVGYQPYRVVRRR

SEQ ID NO: 15 - protein, 71 aa, Artificial Sequence
  ACE2 receptors
EEVIAWNSNNLDSKVGGNYNYLYRCGSGSGQAGSTFGNGVEGFNGYFCLQSYGFQPTNGVGYQPYRVVRRR

SEQ ID NO: 16 - protein, 35 aa, Artificial Sequence
  SARS-CoV-2 spike protein RBD
IEEQAKTFLDKFNHEAEDLFYQSSLASWNYNTNIT

SEQ ID NO: 17 - protein, 77 aa, Artificial Sequence
  SARS-CoV-2 spike protein RBD
IEEQAKTFLDKFNHEAEDLFYQSSLASWNYNTNITEENVQNMNNAGDKWSAFLKEQSTLAQMYPLQEIWDLGKGDFR

SEQ ID NO: 18 - protein, 125 aa, Artificial Sequence
  SARS-CoV-2 spike protein RBD
IEEQAKTFLDKFNHEAEDLFYQSSLASWNYNTNITEENVQNMNNAGDKWSAFLKEQSTLAQMYPLQEIQALTVKLQLQALQQNGSMTQGFWENSMLTDPGNVQKAVCHPTAWDLGKGDFRILMCT

SEQ ID NO: 19 - protein, 141 aa, Artificial Sequence
  SARS-CoV-2 spike protein RBD
IEEQAKTFLDKFNHEAEDLFYQSSLASWNYNTNITEENVQNMNNAGDKWSAFLKEQSTLAQMYPLQEIQALTVKLDPGNVQKAVCHPTAWDLGKGDFRILMCTKVTMDDFLTAHHEMGHIQYDMAYAAQPFLLRNGANEGF

SEQ ID NO: 20 - protein, 15 aa, Artificial Sequence
  SARS-CoV-2 spike protein RBD
SYGFQPTNGVGYQPY

SEQ ID NO: 21 - protein, 36 aa, Artificial Sequence
  2019-nCoV S-HR1(heptad repeat 1)
SLDQINVTFLDLEYEMKKLEEAIKKLEESYIDLKEL

SEQ ID NO: 22 - protein, 38 aa, Artificial Sequence
  SARS-CoV HR1(heptad region)
NGIGVTQNVLYENQKQIANQFNKAISQIQESLTTTSTA

SEQ ID NO: 23 - protein, 19 aa, Artificial Sequence
  SARS-CoV spike protein
MWKTPTLKYFGGFNFSQIL

SEQ ID NO: 24 - protein, 35 aa, Artificial Sequence
  SARS-CoV-2 spike protein
SLDQINVTFLDEYEMKKLEEAIKKLEESYIDLKEL

SEQ ID NO: 25 - protein, 43 aa, Artificial Sequence
  SARS-CoV-2 spike protein
SLDQINVTFLDEYEMKKLEEAIKKLEESYIDLKELPEGCPALM

SEQ ID NO: 26 - protein, 39 aa, Artificial Sequence
  SARS-CoV-2 spike protein
SLDQINVTFLDEYEMKKLEEAIKKLEESYIDLKELPEGC

SEQ ID NO: 27 - protein, 23 aa, Artificial Sequence
  Sequence is synthesized
IEEQAKTFLDKFNHEAEDLFYQS

SEQ ID NO: 28 - protein, 38 aa, Artificial Sequence
  Sequence is synthesized
ESLSLSLSLSLSLEGIEEQAKTFLDKFNHEAEDLFYQS

SEQ ID NO: 29 - protein, 27 aa, Artificial Sequence
  Sequence is synthesized
SALEEQLKTFLDKFMHELEDLLYQLAL

SEQ ID NO: 30 - protein, 43 aa, Artificial Sequence
  Sequence is synthesized
ESLSLSLSLSLSLEGSALEEGQLKTFLDKFMHELEDLLYQLAL

SEQ ID NO: 31 - protein, 43 aa, Artificial Sequence
  Sequence is synthesized
KSLSLSLSLSLSLKGSALEEGQLKTFLDKFMHELEDLLYQLAL

SEQ ID NO: 32 - protein, 19 aa, Artificial Sequence
  Sequence is synthesized
QYKTYIDKNNHYAEDERYK

SEQ ID NO: 33 - protein, 34 aa, Artificial Sequence
  Sequence is synthesized
ESLSLSLSLSLSLEGQYKTYIDKNNHYAEDERYK

SEQ ID NO: 34 - protein, 34 aa, Artificial Sequence
  Sequence is synthesized
KSLSLSLSLSLSLKGQYKTYIDKNNHYAEDERYK

(19) United States
(12) Patent Application Publication
(10) Pub. No.: US 2021/0388031 A1
(43) Pub. Date: Dec. 16, 2021

(54) Designer Peptide Opsonins

(71) Applicant: New Jersey Institute of Technology, Newark, NJ (US)

(72) Inventor: Dr. Vivek A. Kumar, Newark, NJ (US)

(73) Assignee: New Jersey Institute of Technology, Newark, NJ (US)

(21) Appl. No.: 17/348,994

(22) Filed: Jun. 16, 2021

Related U.S. Application Data
(60) Provisional application No. 63/039,780, filed on Jun. 16, 2020.

(51) Int. Cl. C07K 14/005; G16B 15/00

(52) U.S. Cl. CPC C07K 14/005; C07K 2319/735; G16B 15/00

(57) ABSTRACT

Disclosed is a new strategy, composition, and method for a self-assembling peptides (SAP) that will self-assemble atop COVID-19 for immune destruction and act as a functional COVID-19 opsonin. The designed self-assembling peptides bind and assemble atop SARS-CoV-2—providing a new mechanism of infection abrogation. One benefit is avoidance of side effects that other formulations or repurposed drugs contain. Other feature of this technology include: (a) (SAP) are composed of L-amino acids that are highly biocompatible sub-Q, IM and IV (no adverse reaction for 10% blood volume replacement with 1 w % peptide in mice or rats); (b) Highly targeted approach that binds to viral particles preventing side effects; (c) SAP are intrinsically biodegradable and endocytosed; (d) SAP bound COVID-19 may promote engulfment, clearance, and antigen presentation (for immunity development); and (e) targeting the spike protein with SAP may allow treatment of other coronaviruses.

### CROSS-REFERENCE TO RELATED APPLICATIONS

The present application claims the benefit of the filing date of U.S. Provisional Patent Application No. 63/039,780 filed on Jun. 16, 2020 the disclosure of which is hereby incorporated herein by reference.

### STATEMENT REGARDING FEDERALLY SPONSORED RESEARCH

This invention was made with government support under Contract Nos. NIH NEI R15EY029504 and NSF 1903617 awarded by the National Institutes of Health and National Science Foundation, respectively. The government has certain rights in this invention.

### FIELD OF USE

The present application relates to the field of self-assembling peptides with different mimic sequences for a novel strategy, composition, and process to bind to a foreign sequence using a targeting sequence. Specifically, the present application discloses a material that self-assembles on a foreign antigen to prevent binding due to stearic hindrance and/or lack of binding site availability to target the foreign antigen. Further self-assembly atop the antigenic sequence may target that sequence for destruction via phagocytosis. This is similar to how an antibody works to specifically target an antigen and an opsonin works in opsonizing a target antigen for destruction.

### BACKGROUND OF THE INVENTION

In recent times, severe acute respiratory syndrome coronavirus 2 (SARS-Co-V-2), the strain of coronavirus that causes corona virus disease 2019 (COVID-19), has changed the way people live their lives. The challenge faced is to develop a vaccine to immunize in the hope that herd immunity provides added protection, and to develop tailored therapeutics that can treat the afflicted. A number of vaccine candidates are being explored, as is the development of immune memory and advancing understanding of tolerance development—helping explain the potential differences in the cytokine storm in older patients and milder infection in younger patients. With respect to therapeutics, the reduction of viral burden is paramount. Numerous drugs have been repurposed for treatment of COVID-19—most notably the anti-parasite drug hydroxychloroquine and Ebola antiviral Remdesivir®.

Though “shots in the dark”, these therapeutics and a handful of other non-specific therapies have fortunately shown some clinical benefit, however, not without side effects and related mortality. Furthermore, attempts to develop drugs capable of binding to specific domains of the virus, and targeting them for destruction to emulate the nature of antibodies the body produces have been met with limited success. Even though the process of tagging the viral protein oat (spike protein) similar to antibodies that the body generates may be a path for a therapeutic response to COVID-19, however, peptides and other small molecule drugs that bind and prevent viral fusion are known to have poor shelf lives and in vivo stability making this option less attractive in the past.

Therefore, there exists a critical need for a novel therapeutic strategy that allows for effective therapeutic treatment of COVID-19 without the negative side effects and related mortality of current methodologies. In addition, there is a need for peptide-based drugs that have improved shelf lives and in vivo stability to remain effective. Finally, this strategy may be expanded to other coronaviruses with similar homology, or other antigenic sequences for opsonization of other antigens. These may be of viral, bacterial or other (potentially mammalian) origin.

### SUMMARY OF THE INVENTION

Disclosed is a newly developed self-assembling peptide platform that consists of a self-assembling domain and an antigenic binding domain. Compared to the above prior attempts, the presently disclosed composition and method solves the problems of current state of the art, meets the above requirements, and provides many more benefits. The present disclosure is a rationalized approach to develop drugs capable of binding to specific domains/antigenic sequences. Focused in this disclosure are antigenic sequences of viral origin and targeting them for destruction—emulating the native antibodies the body produces; designed specifically for COVID-19.

In one aspect, disclosed is a novel strategy to bind to a foreign sequence using a targeting sequence. The sequence includes, for example, but is not limited to antigenic sequences proposed by several groups in table 1 and 2 below. The function of the targeting sequence is similar in function to an antibody that the body produces. Depending on the implementation, the targeting sequence may self-assemble on this foreign antigen similar to complement or antibodies in the human body. The targeting sequence would prevent binding of the foreign sequence to the body's cells due to stearic hindrance and/or lack of binding site availability. Self-assembly of the self-assembling domain portion of this peptide will allow epi-viral self-assembly. Self-assembly of the peptide into nanofibers atop the virus. Self-assembled peptide nanofibers have been shown to be phagocytosed in vivo. Thus the binding of the targeting sequence would target for destruction the foreign sequence/virus, due to blocking of binding and epi-viral self-assembly. This is as such as but not limited to the strain that causes COVID-19, via phagocytosis like an opsonin. Notwithstanding, the approach proposed is that of a platform and can be facilely adapted to a growing list of peptide inhibitor sequences as as shown in Table 2.

In another aspect, a self-assembling peptide (SAP) backbone is utilized that stabilizes peptides. The objective is to bind anti-viral or anti-bacterial or anti-antigenic short peptides to the SAP, thereby stabilizing them for in vivo use. In this embodiment, it is proposed binding a SARS-CoV2 antigenic sequence to the SAP—stabilizing it and allowing for epi-viral self-assembly atop SAR-CoV2—and thus abrogation of infection. This may this be a treatment for COVID-19. SAP forms nanofibers that are endocytosed into vacuoles (cleared) in vivo. It is hypothesized that SAP can be functionalized for binding to antigenic sequences, as in this embodiment to SARS-CoV-2 preventing infection. Thus, further, it is hypothesized that SAP will self-assemble atop COVID-19 and target it for immune destruction. As such the primary invention is a functional COVID-19 opsonin: i) specifically binds COVID-19 due to recognition of a specific domain on the virus; and ii) self-assembles on the surface of the virus promoting its clearance (phagocytosis) by the immune system.

In yet another aspect, the methodology includes first to synthesize and determine if antigenic sequences from SARS-CoV-2 and SAP-conjugate peptides have in vitro binding to demonstrate utility as a drug. The second aim is designed to take the first steps towards translation of this specific peptide through a dose range finding study in rodents. Animals will be challenged with different concentrations of therapeutic peptides IV and orally. This will aim to determine the maximum tolerable dose and solubility limit dose that can be administered in animals, benchmarking human dosing. Upon successful completion of these studies, 2 species toxicity and Good Manufacturing Procedures (GMP) manufacturing remain prior to beginning first-in-human trials. The objective is to rapidly bring this therapy to patients in approximately 12-24 months as a highly targeted approach with potential broad applicability to other coronaviruses, and avoiding side effects of other formulations or repurposed drugs; and broadening scientific investigation into the supramolecular interaction of functionalized SAP.

The present invention also shows for the first time a peptide that binds to a target (shown herein through enzyme-linked immunosorbent assays (ELISA) and surface plasmon resonance (SPR) assays), and doesn't let go of the target or foreign substance (shown herein through SPR analysis), self-assembles on that target (shown herein through atomic force microscopy (AFM) and scanning electron microscope (SEM) analysis), and causes the target to be destroyed through phagocytosis.

Depending on the embodiment, a variety of other ligand sequences that target other protein domains in COVID-19, other spike proteins in other coronaviruses, other viruses or bacteria or pathogens may be utilized with the concepts of the present invention. The potential generation of new drugs at a significantly lower cost compared to growth factor or monoclonal antibody therapeutics may prove to be a new paradigm in drug design and targeting—opening avenues for rapid large-scale production. Peptide synthesis is almost ubiquitous worldwide, with amenability to conventional drug fill and finish processes.

Using the concepts of the present invention, further advances in synthetic procedures allows the rapid generation of gram-kg scale of this μg-mg dosed drug. Since these are specific to a virus/foreign antigen, at least the following variations may also utilize the concepts of the present invention in: 1) use as IV therapeutics 2) use as intranasal therapeutics 3) use as oral, subcutaneous (sub-Q) or intramuscular (IM) therapeutics 4) use as diagnostic (akin to a capture antibody) and 5) use as a hydrogels/lyophilized mesh to trap COVID/pathogens in personal protective equipment (PPE).

Another advantage of the present invention is providing a self-assembling peptide (SAP) that will self-assemble atop COVID-19 and target it for immune destruction, acting as a so-called functional COVID-19 opsonin. The present invention brings this therapy a step closer to patients as a highly targeted approach with potential broad applicability to other coronaviruses. The present invention avoids side effects that are common with other formulations or repurposed drugs.

In yet another advantage of the present invention is development of a robust targeted therapeutic with easily adaptable large scale manufacturing capability, ii) establish a delivery route with immediate and demonstrable prophylactic or post-exposure COVID-19 protection, iii) develop a tunable pan-coronavirus platform for drug design and targeting of variants and the next pandemic. Depending on the implementation, a rationalized approach is utilized using a peptide design by using BUDE alanine scanning, SWISS-Model, Rosetta, and binding stability using molecular dynamics (MD) to facilitate the development of peptides with novel pan-coronavirus functionality, and address the current (SARS-CoV-2), previous (SARS), and other coronaviruses, for example including, but not limited to, MERS, HCoV-229E, HCoV-NL63, HCoV-OC43, and HCoV-HKU1) and future pandemics. The ability of the SAP domain to guide assembly atop viral particles (visualized with Co-I Prasad), potentially clustering atop antigenic determinants, and allow for the opsonization of viral targets is accomplished using the present invention.

The present invention also provide a functionalized self-assembling peptide (SAP) that can bind the SARS-CoV-2-ACE2 receptor binding domain of spike protein, thereby preventing infection, and can self-assemble atop the virion, thereby promoting opsonization. Further the present invention allows facile tuning of the peptide sequence through rationalized computational design that allows pan-coronavirus targeting for other variants.

Some other advantages and features that differentiate the present invention over current drugs, short inhibitor peptides or other delivery particles are: (a) the present invention's self-assembling peptides (SAP) are composed of L-amino acids that are highly biocompatible with drug deliveries given sub-Q, IM and IV. As disclosed herein there were no adverse reactions for 10% blood volume replacement with 1 w % peptide in mice or rats—10 mg; (b) highly targeted approach in the present invention provides binding of the SAPs to viral particles thereby preventing side effects; (c) SAP are intrinsically biodegradable and endocytosed; (d) SAP bound SARS-CoV-2 molecules/self-assembled nanofibers may promote engulfment, clearance, and antigen presentation (for immunity development); and (e) targeting the spike protein with SAP may allow treatment of other coronaviruses.

Depending on the embodiment, through iterative computational design, the invention includes identification of a 19-mer peptide that binds in silico to SARS-CoV-2 and SARS with high specificity. Facile modification with a self-assembling peptide (SAP) platform results in a new sequence ESBP3 [SEQ. 33]. Through live virus infection of Vero cells in vitro assays, ESBP3 demonstrates 10 nM IC₅₀ inhibition, as well as a robust rodent in vivo intravenous (IV) safety profile (at mM daily dosing for 10 days). Realizing the therapeutic potential of this approach for clinical translation, developed was a peptide drug platform as a treatment modality for ongoing COVID and future pan-coronavirus pandemics.

One objective of the invention is to evaluate in vivo efficacy and further alter existing SARS-CoV-2 binding peptide (if necessary) to target SARS and other coronaviruses as more structural data of the virus(es) become available. Specifically, the invention further utilizes the following hypothesis: 1) a computationally identified SARS-CoV-2 spike protein-binding peptide conjugated to the SAP domain will prevent infection, 2) subsequent association of free monomers to the bound peptide:SARS-CoV-2 complex will promote further secondary self-assembly (guided by the added SAP domain) atop the virion, promoting opsonization; and 3) facile tuning of the peptide sequence through rationalized computational design will facilitate the generation of materials with broad pan-coronavirus targeting.

The above objects and advantages are met by the present invention. In addition the above and yet other objects and advantages of the present invention will become apparent from the hereinafter-set forth Brief Description of the Drawings, Detailed Description of the Invention and claims appended herewith. These features and other features are described and shown in the following drawings and detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

So that those having ordinary skill in the art will have a better understanding of how to make and use the disclosed composition and methods, reference is made to the accompanying figures wherein:

FIG. 1 is a schematic of one embodiment of the proposed self-assembling peptide opsonin strategy;

FIGS. 2A-2F are photographs and graphs showing an embodiment of the design of the self-assembling peptide (SAP);

FIGS. 3A-3K are photomicrographs and charts showing biocompatibility and intracellular trafficking utilizing the concepts of the present invention;

FIGS. 4A-4D are graphs and illustrations showing super stoichiometric binding used to target proteins;

FIGS. 5A-5F are illustrations showing workflow for a computational model utilizing the concepts of the present invention;

FIGS. 6A-6D are charts and graphs showing preliminary safety and efficacy information of ESBP2 utilizing the concepts of the present invention; and

FIGS. 7A-7D are photomicrographs, charts, and illustrations showing structural analysis of ESBP3 utilizing the concepts of the present invention.

### DETAILED DESCRIPTION

The present disclosure is directed to a new composition, a process, and novel strategy. A major concern for peptide-based therapeutics is the stability of peptides in vivo. A series of modifications with polymeric, including but not limited to polyethylene glycol (PEG), dendrimer, poly(lactic-co-glycolic acid) (PLGA), liposome, and the like, carriers allow for improved stability of peptides. However, non-covalent interactions of peptides leave exposure to proteolysis and degradation in minutes to hours for most oral, IV or even Sub-Q peptides. Circumventing this, SAP offers a peptide-based platform for the non-covalent stabilization of peptides. Through supramolecular interactions (hydrogen bonding, van der Waals interactions and ionic interactions) disclosed herein is a platform that spontaneously forms β-sheet based nanofibers in aqueous solution. It is demonstrated here that this SAP platform for ligand presentation provides features that allow the formation of peptides nanofibers that stabilize peptide inhibitors, bind and block SARS-CoV-2, and opsonize the virus targeting it for immune destruction.

In one embodiment, the present compositions, methods, and strategy works as follows. A self-assembling peptide (SAP) is provided that can specifically target a pathogen, and promote its own endocytosis behaving as an opsonin. Again, this may have utility as a therapeutic against COVID-19, and various other pathogens. In the example of COVID-19, specificity is conferred by conjugating a ligand sequence (LS) that binds a domain on COVID-19 (such as the Spike protein). SAP have been shown to be phagocytosed into cellular vacuoles that are degraded. Through phagocytic clearance in vacuoles, potential presentation on MHC II for immunization (development of antibodies against SARS-CoV-2) is possible, thereby proving potentially additional utility of the platform. These synthetic materials (SAP-LS) act like antibodies in that they specifically bind a target and signal the immune system to destroy that target to deliver opsonization.

These new synthetic materials (SAP-LS) act like complement proteins that bind pathogens, and self-assemble on their surface to form nanofibers and signal the immune system to destroy that target and hence provide opsonization like features. This immune system destruction, depending on the embodiment, could be through phagocytosis or secretion of enzymes or both depending on the embodiment. The SAP-LS peptides will be able to bind SARS-CoV-2 or other pathogens based on LS domains, and self-assemble on their surface targeting them for immune destruction (SAP domain).

Shown here is an investigation study into a COVID-19 binding LS domain termed SBP1 (and others) and SAPs. In developing this platform studied is the ligands that have specificity for COVID-19 such as the SARS-CoV-2-ACE2-receptor binding domain (RBD) SBP1 peptide and others, for binding kinetics, and subsequent epi-viral self-assembly.

These kinetic studies of functionalized SAP binding peptides to specific targets will select targets for in vitro efficacy evaluation. In vitro efficacy using SAP versus diluted peptides, may elucidate unique mechanisms of peptide-COVID spike protein RBD binding. The ability for these specific targeting materials to bind and self-assemble on the virion surface and potentially target them for immune destruction is a unique approach that goes beyond blocking of binding using decoy receptor or mimicking proteins (peptide inhibitors such as SBP1 alone).

Also shown and described herein is a proposed set of experiments that will determine the potential for SAP to bind and fibrillize on the surface of virions, opsonizing them for endocytosis. Together, the studies investigated for both SAP-ligand constructs, will investigate possibilities in opsonization, potential immunity development and application against other coronaviruses, and potentially a proof of concept platform for other pathologies.

Characterized herein is a hamster model of COVID-19, owing to ACE-2 abundance and similarity of disease avoiding contrived humanized mouse models. Evaluated is the efficacy of these peptides and other relevant clinical strategies to treat COVID-19 in hamsters as a therapeutic screening model. Below is a summary of the study proposed.

### SUMMARY OF EXPERIMENTATION

Numerous drugs have been repurposed for treatment of COVID-19—most notably the anti-parasite drug hydroxychloroquine and Ebola antiviral Remdesivir®. Though “shots in the dark”, these and a handful of other non-specific therapies have fortunately shown some clinical benefit—not without side effects and related mortality. This begs a rationalized approach for the development and testing of drugs capable of treating COVID-19. Accordingly, peptides (and other small molecule drugs) that bind and prevent viral fusion are being developed but lack adequate animal models to rapidly characterize viral load diminution and therapeutic efficacy. Building upon the former, engineered herein are peptide sequences that bind to specific domains of the virus, targeting them for destruction—emulating antibodies the body produces. This process of tagging the viral protein coat (spike protein) can now be realized thanks to the recent publication of the 3D structure of the virus. Unfortunately, peptide based drugs are known to have poor shelf lives and in vivo stability. Shown herein are engineered a self-assembling peptide (SAP) backbone that stabilizes peptides. It is planned to bind a recently discovered Spike-protein binding peptide to the present SAP, thereby stabilizing it for in vivo use. With respect to animal models, there is a lack of rodent models to efficiently screen therapeutics, with mouse infections requiring humanized/genetically engineered strains, or use of mouse-adapted SARS-CoV-2 strains that do not replicate human pathophysiology. To this end, optimize is a golden Syrian Hamster model of SARS-CoV-2 infection to exhibit clinical manifestation like humans. Similar reports from a few other groups bolster the use of this rapid screening model which exhibits ACE-2 mediated infection similar to humans unlike naïve mice that uniquely exhibit central nervous system deficit, nonhuman primates with species variant viral replication/pathology and high cost, or poor recapitulation in rabbits/ferrets.

It is planned to address a significant gap between repurposed “attempts”, and a rationalized approach to developing targeted therapeutics against COVID-19. It is hypothesized that SAP can be functionalized for binding to SARS-CoV-2 preventing infection, and evaluated for dosing and efficacy using golden Syrian Hamster model. Specific to the former: it is sub-hypothesized that SAP will self-assemble atop COVID-19 and target it for immune destruction. Specific to the latter: it is further sub-hypothesize a golden Syrian Hamster model will mimic human manifestations of the infection and allow rapid screening of peptides and small molecule therapeutics. As such an objective is to develop a functional COVID-19 opsonin evaluated for efficacy in a benchmarked Syrian hamster model.

Two further objectives are explored. First is to study SAP in vitro binding, cytocompatibility, and efficacy. Second is to evaluate the utility of golden Syrian Hamster model in mimicking human COVID-19 symptoms, spread and therapeutic screening potential. Animals will be challenged with therapeutics, that include but are not limited to, antiviral medication Remdesivir® (C₂₇H₃₅N₆O₈P), IL-6 inhibitor (Tocilizumab) and SAP, to validate clinical findings, explore symptom mitigation and potentially provide a targeted cure, respectively. This therapy builds upon a highly targeted approach with potential broad applicability to other coronaviruses, thereby avoiding side effects of other formulations or repurposed drugs. Also, in models it helps to create the translational bridge from in vitro efficacy to in vivo efficacy and first in human studies.

### Examples

To facilitate a better understanding of the present invention, the following example of specific instances is given. In no way should the following example be read to limit or define the entire scope of the invention. The following materials and methods were employed for the Example below.

There are at least two significant gaps in current therapy for COVID-19 and the field in general that need to be immediately addressed and are addressed by the present disclosure: i) the lack of a targeted stable therapeutic designed to uniquely treat COVID-19, and ii) a suitable therapeutic screening (small) animal model that can mimic human disease.

Shown and described herein is the development of a class of tunable self-assembling peptides (SAP) that may prove useful in binding to COVID-19 and targeting it for immune destruction. Present investigator experience in a golden Syrian hamster models (3 to 6 months) known to have similar viral infectivity due to constitutive ACE-2 expression on lung epithelial cells compared to (the lack thereof) in other rodents assists in the investigation. This allows for disease mimicry (clinical signs/change in physical activity), understanding mechanisms of viral infectivity, and evaluation (screening) of therapeutic candidates.

The present COVID-19 binding platform offers four key advantages and innovations to enhance the armamentarium against COVID-19. First, a proven targeting strategy that is specific for the spike protein and SAP stabilized for in vivo delivery and long term degradation (1-4 weeks). Second, the potential for this low cost self-assembling opsonin to be efficacious against other coronaviruses, as well as the treatment of latent (re)infection. Third, an established manufacturing scheme (lab and peptide API manufacturer scale). Finally, a wealth of preliminary data on the SAP platform establishing high dose tolerability in mice, rats and dogs priming for translation.

To this extent, proposed and described below is an aggressive 2 year plan to develop the peptide (Aim 1) and establish efficacy in the present in vivo model (Aim 2). The specific hypothesis is that functionalized SAP can bind SARS-CoV-2-ACE-2-RBD preventing infection, as a potential means of opsonization; that can be screened alongside other therapeutic candidates in a characterized golden Syrian hamster model that mimics the pathologic sequelae in humans. Through the course of the following 2 aims, investigated is the potential mechanism and kinetics of SAP and its efficacy compared to current therapeutics in development/market in its ability to prevent in vivo infection.

Aim 1: Characterization of a functionalized peptide that inhibits SARS-CoV-2. Evaluation of novel epi-viral self-assembly to opsonize the virus.

Synthesis and stability of novel peptides is optimized to target low cost (<$1/dose) and large (1-100 g) scale at >85% purity. Binding kinetics determined using SPR of candidate peptides will be compared to rhACE-2 to optimize peptide design and eliminate poor binding candidates. Negatively stained TEM will be used to visualize peptide virion binding and potential epi-viral fibrillation/opsonization. In vitro efficacy is determined using the Caco-SARS-CoV-2 infection and cytotoxicity to determine efficacious and cytotoxic concentrations, EC50, CC50, respectively. Success is determined by: i) stable and scalable generation of SAP, ii) superior in vitro receptor binding kinetics to rhACE-2 iii) attempted visualization of opsonization); iv) determination of in vitro efficacy of SAPs.

Aim 2: Characterization of a small animal model to screen therapeutic candidates against SARS-CoV-2. Evaluation of model's ability to mimic human viral infection and screen therapeutic candidates.

The utility of small animal screening model is measured using FDA approved Remdesivir®, cytokine storm mitigating IL6 inhibitor (Tocilizumab), and a targeted SAP from Aim 1. Screening of existing therapeutic candidates against SARS-CoV-2 will establish the utility of this model, with complimentary screening of targets identified in Aim 1. Success is measured by: i) the ability to mimic the human disease phenotype depending on viral dose response; ii) evaluate communal spread, iii) allowing evaluation of therapeutic candidates; iv) benchmark human dosing of SAP from Aim 1.

Aim 3: Mechanism—Understanding SAP anti-viral drug action. Preliminary (Prel.) data have demonstrated that ESBP3 binds and inhibits live virus (isolate USA-WA1/2020) infection. These novel materials were challenged against B.1.1.7, B.1.351, and P.1 strains (BEI Resources Inc) to demonstrate the facile and adaptable nature of this platform, and confirm in silico inhibition. Further, TOR2/Wildtype strain/SARS-HCoV is sourced and tested in vitro efficacy. An opsonization mechanism is investigated through SPR competitive binding and EM (Co-I Prasad). TEM analysis of viral titers preincubated with several SAP opsonin to visualize assembly atop a virus. Finally, testing in a Golden Syrian Hamster model is done to establish in vivo efficacy.

Aim 4: Translation—Optimizing intranasal delivery, large animal PK, facile and scalable SAP GMP manufacturing. Prel. data show IV safety at daily dosing up to 10 days, facile >90% purity non-GMP gram-scale syntheses, and robust analytical/plasma PK LC/MS method development. ESBP3 intranasal delivery shown in mice is evaluated with evaluation in large animal (canine) to follow. Tissue homogenates is used to determine intranasal and IV ESBP3 via LC/MS analysis.

Aim 5: Platform—Computational design of a pan-coronavirus targeting SAP. Prel. data show ESBP3 targets SARS-CoV-2, SARS and other coronaviruses due to their epi-viral self-assembly. First, the multivalency afforded by functionalized SAP targeting may explain enhanced SBP3 binding, which will be modeled in silico using Rosetta and MD simulations. NMR assists in guiding SAP assembly constraints in MD simulations. Next, the sequence is altered to identify a canonical sequence to bind with SARS-CoV-2 variants: B.1.1.7, B.1.351 and P.1; and MERS, SARS-HCoV, HCoV-229E, HCoV-NL63, HCoV-OC43, and HCoV-HKU1 coronaviruses; and subsequently develop hetero-functional pan-coronavirus drugs.

It is envisioned that these studies will help create a new class of targeted SAP therapeutics with potential expandability into other (corona)viruses; that can be rapidly evaluated in a human-mimicking small animal screening model that will help determine efficacy and dosing, and a potential mechanism of action in vivo. For clinical translation, it is envisioned facile GMP manufacturing will complement FDA (drug or breakthrough drug designation) fast-tracking towards first-in-human trials.

This project addresses an important problem in the field of developing targeted therapeutics and addresses a critical barrier due to the lack of an in vivo scalable small animal model to screen therapeutics. Many pharmaceutical small molecule drugs, antibodies, and non-conventional therapies are currently being employed in the fight against COVID-19.²⁻¹⁶ The development of a vaccine may offer protection, but a complementary pharmacologic therapeutic approach can affect treatment more immediately.

Recent studies suggest hydroxychloroquine, azithromycin and other repurposed drugs may prove useful, although the mechanism of action is unknown and side-effects and mortalities have occurred.^{6,14,17,18} Due to the structural similarity of SARS-CoV and SARS-CoV-2, anti-viral effective against SARS could be worthwhile in treating severe COVID-19 cases. Recently, Remdesivir®, an adenosine nucleotide analog shelved against Ebola, showed some benefit against SARS-CoV-2, similarly to Lopinavir-Ritonavir.^{15,17,19-22} These non-specific approaches against COVID-19 show some clinical benefit.^{14,15,23,24} While beneficial in the immediate management of the pandemic, again side-effect profiles from these non-specific approaches leave a significant gap in the design of targeted therapeutics against SARS-CoV-2.

The cryo-EM structure of COVID-19's ACE-2 binding spike protein was recently published,²⁵ leading to rationally designed and tested drugs against the spike protein.^{1,26-28} The prior research on targeted therapeutics and the design of small molecule, antibody, and peptide-based drugs against the spike protein and receptor binding domain (RBD) of COVID-19 is an approach that has roots in the body's native antibody defense mechanisms.^{1,25-29} The sequence homology between coronaviruses (9-20 nucleotide difference between SARS and COVID-19) highlights the conserved RBD that remains a viable target for rationalized approaches to have broadly neutralizing ability.^{30,31}

A 23 amino acid peptide within human ACE-2 that is responsible for binding to COVID-19 has been identified.¹ Molecular dynamic simulations show SBP1: IEEQAKTFLDKFNHEAEDLFYQS [SEQ. 2], a sequence endogenous to ACE-2, binds with nM affinity to SARS-CoV-2-RBD. This strategy may allow for the formation of a 3D binding pocket for targeted inhibition of COVID-19 spike protein binding with its canonical receptor, which small molecule inhibitors may not be able to fully inhibit.³² Emblematic of this is the peptide based Enfuvirtide® that inhibits HIV viral entry by binding to viral protein gp41.³³ However, poor peptide stability requires 2× daily injections and adjunctive HAART to maintain low viral load.^{32,33} Specific to COVID-19, SBP1 can bind and tag the virus—preventing it from binding to human lung epithelial ACE-2.¹ Clinically, soluble recombinant hACE2 and virus specific antibodies from convalescent patient plasma have been explored for opsonizing the virion.^{9,34-38} However, a peptide-based approach is preferable, due to the possibility of blood-borne disease, the availability and the cost of recombinant proteins.^{39,40}

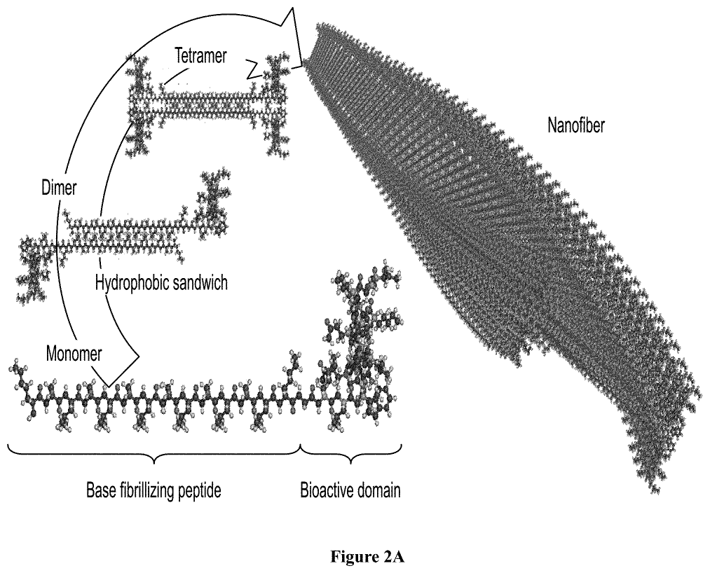

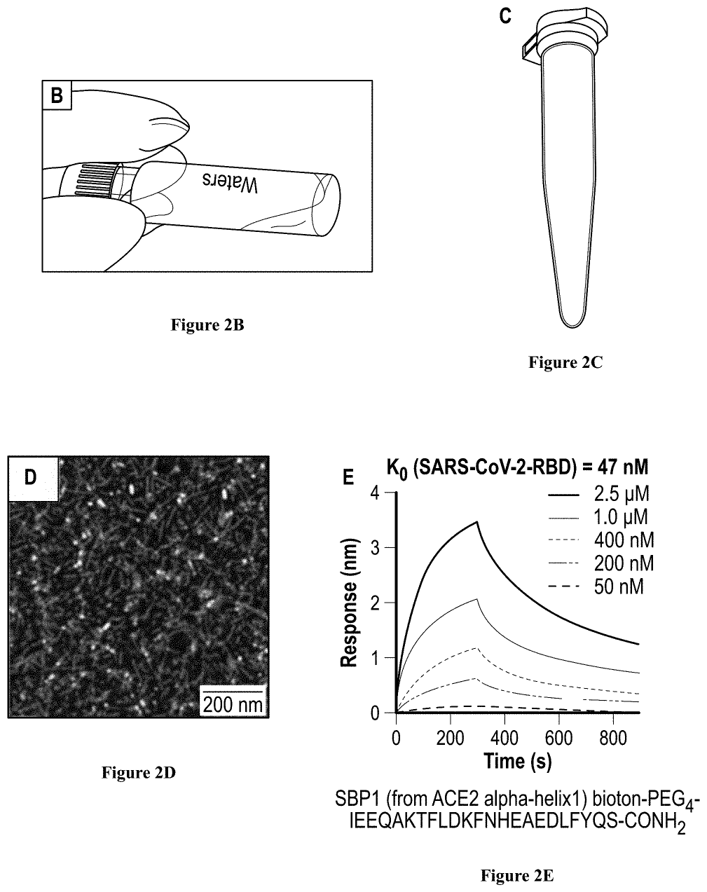

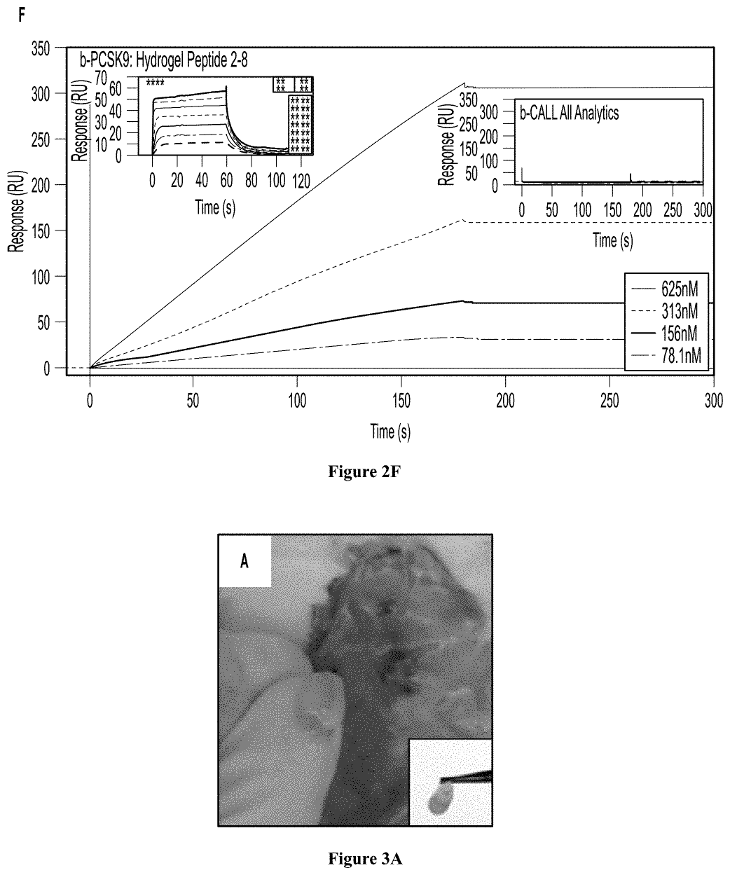

The aforementioned poor peptide stability is a major concern for peptide-based therapeutics in vivo,³⁹⁻⁴² which can be improved by modifications with polymeric carriers.^{1,39-43} Non-covalent interactions leave most peptides exposed to proteolysis and degradation within minutes to hours, whether they were administered orally, intravenously, or even subcutaneously.^{39-41,44-46} SAPs circumvent this issue through non-covalent stabilization, and supramolecular interactions.^{4,7-51} The present inventor has developed a platform that spontaneously forms β-sheet based nanofibers in aqueous solution (FIG. 2).⁴⁸⁻⁵¹ Additional features that make this SAP platform ideal for SBP1 presentation are outlined in the preliminary data and FIG. 2 below, including viral opsonization.

Importantly, in vitro studies do not replicate the challenges in stability, metabolism, transport, delivery, and efficacy determined in in vivo studies. Compounding this is the lack of understanding of the pathogenesis and host-response in a suitable animal model. Prior research on targeted small animal models demonstrates failures in rat or mouse models to replicate clinical features such as viral load in blood and various organs, pathological evidence, and cytokines levels during infection that would facilitate the rationalized design and evaluation of therapeutics. The lack of a specific animal efficacy model is addressed partially with mouse infections requiring genetically engineered strains, or the use of mouse-adapted SARS-CoV-2 strains that do not recapitulate human clinical features—principally due to the lack of constitutive ACE-2 expression. Capitalized was the present investigator's in vivo expertise with a golden Syrian hamster model that constitutively expresses high levels of ACE-2, and exhibits human-like clinical symptoms, in order to evaluate dosing and safety. If achieved a targeted therapeutic can be developed to abrogate COVID-19 symptoms, and establish a robust human pathophysiological manifestation rodent model to screen therapeutics. These in vivo models can further answer key outstanding COVID-19 questions: i) understanding neutralizing antibodies against re-infection; ii) spread from recovered animals; iii) study of IL-6 inhibitors such as Tocilizumab® to mitigate the cytokine storm.

These present significant gaps that will be addressed herein by in vitro models, and animal dosing safety, prior to human trials. Successful completion will result in the development of SAP therapeutic platform with demonstrable efficacy in a fit-for-purpose rodent model, as well as the potential for translatability of epi-viral self-assembly to attack other (corona)viruses.

Again, developed herein is a self-assembling peptide that can specifically target a pathogen, and promote its own endocytosis behaving as an opsonin. In developing this platform studied is the ligand (SARS-CoV-2-RBD) SBP1 peptides' binding kinetics, and subsequent self-assembly.^{1,25,52,53}

Importantly, the present disclosure's ability for these opsonin-like materials to assemble on the virion surface and potentially target them for immune destruction is a unique approach that goes beyond blocking of binding using decoy receptor or mimicking proteins (SBP1 alone). The proposed set of experiments determine the potential for SAP to bind and fibrillize on the surface of virions, opsonizing them for endocytosis. Through phagocytic clearance in vacuoles, potential presentation on MHC II for immunization is possible. Together, the studies investigated for both SBP1 (self) peptide, and non-immunogenic SAP, investigate possibilities in opsonization and application against other coronaviruses,^{6,54} and potentially a proof of concept platform for other pathologies. The concept of epi-viral self-assembly, designer opsonin development and use of SAP to achieve these effects is a novel platform with broad applicability. Complementing this is the unique information learnt from the custom golden Syrian Hamster model that will provide unique insight on infectivity and therapeutic efficacy/screening.

More broadly, the disclosed will increase the utility of self-assembling peptides to act as scaffolds for drug delivery and as drugs themselves.^{1,53,54,57,60,62,65} At a fundamental level, the disclosed will create a deeper understanding of the peptides and techniques governing self-assembly and signaling, ultimately helping the scientific community's understanding of this versatile SAP platform.

Furthermore, the potential inexpensive generation of new drugs may prove to be a significant refinement of the approaches in drug design and targeting—opening avenues for rapid large-scale production for peptide synthesis, with amenability to conventional drug fill and finish processes. Further advances in synthetic procedures allows the rapid generation of gram-kg scale of this μg-mg dosed drug. This approach is supported by the present investigator's recent publications in material characterization, receptor binding kinetics, in vitro biocompatibility and in vivo compatibility/efficacy.^{40,47-51,55-70}

To-date the golden Syrian hamster model for SARS-CoV-2 has not been used for therapeutic evaluation. The proposed systematic investigation will strive to emulate the symptomatic presentation, viral infectivity mechanism, and communal spread. Investigated also is the window of transmissibility from infected hamsters, addressing concerns such as reactivation and protective immune response to future infection including cross-protection between isolates.

The results of this study ill reveal hidden characteristics of COVID-19 such as the extent of extrapulmonary infection and the cytokines involved in disease severity. Ultimately, this model addresses the in vivo animal efficacy gap by using hamsters, allowing wider screening and mechanistic understanding of therapeutic strategies humans. To determine the efficient usage of appropriate therapeutic agents against SARS-CoV-2, based on the available data, selected was potential therapeutics for COVID-19 such as Remdesivir®, IL-6 inhibitors and SAP peptides from Aim 1 for evaluation in this hamster model for the treatment and prevention of COVID-19.⁷¹

In this example, explored was the attachment of the following to a self-assemble peptide framework for possible therapeutic application. The below peptide sequences were studied as shown in Table 1, Table 2, and Table 3. Certain sequences are N-terminally acetylated and C-terminally amidated. SBP1 (denoted as ¹) is known to bind the SARS-CoV-2-RBD. The scrambled sequence (denoted as ²) was used as a control. Other controls include the standalone base SAPs (K2 and E2, denoted as ³). Multiple functionalized SAP-SBP1 peptides are explored (denoted as ⁴) in the below Table 1.

TABLE 1
Sequences of Peptides Studied

| | | | SEQ. |
|---|---|---|---|
| Name | Function | Sequence | NO. |
| KSL₆K | Base- | KSLSLSLSLSLSLK or | SEQ. |
| | Fibril- | K-(SL)₆-K | 1 |
| | lizing | | |
| | Domain | | |
| SBP1¹ | SBP1 | IEEQAKTFLDKFNHEAEDLFYQS¹ | SEQ. |
| | Domain¹ | | 2 |
| SBP1sc² | SBP1 | YIQESEFQLADKETAFELHDNKF² | SEQ. |
| | Domain¹ | | 3 |
| K2³ | Self- | KK-(SL)₆-KK³ | SEQ. |
| | Assembling | | 4 |
| | Peptide³ | | |
| E2³ | Self- | EE-(SL)₆-EE³ | SEQ. |
| | Assembling | | 5 |
| | Peptide³ | | |
| R2³ | Self- | RR-(SL)₆-RR³ | SEQ. |
| | Assembling | | 6 |
| | Peptide³ | | |
| D2³ | Self- | DD-(SL)₆-DD³ | SEQ. |
| | Assembling | | 7 |
| | Peptide | | |
| | (SAP)³ | | |
| K2- | SAP³-SBP1¹ | KK-(SL)₆-KK-G-³ | SEQ. |
| SBP1⁴ | Conju- | IEEQAKTFLDKFNHEAEDLFYQS¹ | 8 |
| | gates⁴ | | |
| E2- | SAP³-SBP1¹ | EE-(SL)₆-EE-G-³ | SEQ. |
| SBP1⁴ | Conju- | IEEQAKTFLDKFNHEAEDLFYQS¹ | 9 |
| | gates⁴ | | |
| R2- | SAP³-SBP1¹ | RR-(SL)₆-RR-G-³ | SEQ. |
| SBP1⁴ | Conju- | IEEQAKTFLDKFNHEAEDLFYQS¹ | 10 |
| | gates⁴ | | |
| D2- | SAP³-SBP1¹ | DD-(SL)₆-DD-G-³ | SEQ. |
| SBP1⁴ | Conju- | IEEQAKTFLDKFNHEAEDLFYQS¹ | 11 |
| | gates⁴ | | |
| KSL₆K- | Mimic | KSLSLSLSLSLSLK-G- | SEQ. |
| G- | Base- | | 12 |
| | Fibril- | | |
| | lizing | | |
| | Domain | | |
| | with | | |
| | glycol | | |
| | spacer | | |

TABLE 2
Additional Peptide Sequences for Binding to
Antigenic Sequences
The sequences shown in Table 2 may be com-
bined with any one of the mimic base
fibrilizing domain and glycol spacer
such as [SEQ. 8]-[SEQ. 12], or [SEQ.
1], [SEQ. 4]-[SEQ. 7] with the
addition of a glycol spacer.

| | | | SEQ. |
|---|---|---|---|
| Name | Amino Acid Sequence | Target | NO. |
| SEQ_10 | EEVIAWNSNNLDSKVGGNYNYLYRCF | ACE2 | SEQ. |
| [3] | RKSNLKPFERDISTEIYQAGSTPGNG | recep- | 13 |
| | VEGFNGYFCLQSYGFQPTNGVGYQPY | tors | |
| | RVVRRR | | |
| SEQ_11 | EEVIAWNSNNDSKVGGNYNYLYRCKM | ACE2 | SEQ. |
| [3] | SECVLGQSKRVQALLFNKVTLAGFNG | recep- | 14 |
| | YFCLQSYGFQPTNGVGYQPYRVVRRR | tors | |
| SEQ_12 | EEVIAWNSNNLDSKVGGNYNYLYRCG | ACE2 | SEQ. |
| [3] | SGSGQAGSTFGNGVEGFNGYFCLQSY | recep- | 15 |
| | GFQPTNGVGYQPYRVVRRR | tors | |
| Inhib- | I EEQA KTFLD KFNHE AEDLF | SARS- | SEQ. |
| itor | YQSSL ASWNY NTNIT | CoV-2 | 16 |
| 1 [4] | | s.pro- | |
| | | tein | |
| | | RBD | |
| Inhib- | I EEQA KTFLD KFNHE AEDLF | SARS- | SEQ. |
| itor | YQSSL ASWNY NTNIT EENVQ | CoV-2 | 17 |
| 2 [4] | NMNNA GDKWS AFLKE QSTLA | s.pro- | |
| | QMYPL QEIWD LGKGD FR | tein | |
| | | RBD | |
| Inhib- | I EEQA KTFLD KFNHE AEDLF | SARS- | SEQ. |
| itor | YQSSL ASWNY NTNIT EENVQ | CoV-2 | 18 |
| 3 [4] | NMNNA GDKWS AFLKE QSTLA | s.pro- | |
| | QMYPL QEIQA LTVKL QLQAL | tein | |
| | QQNGS MTQ GFWEN SMLTD | RBD | |
| | PGNVQ KAVCH PTAWD LGKGD | | |
| | FRILM CT | | |
| Inhib- | I EEQA KTFLD KFNHE AEDLF | SARS- | SEQ. |
| itor | YQSSL ASWNY NTNIT EENVQ | CoV-2 | 19 |
| 4 [4] | NMNNA GDKWS AFLKE QSTLA | s.pro- | |
| | QMYPL QEIQA LTVKLD PGNVQ | tein | |
| | KAVCH PTAWD LGKGD FRILM | RBD | |
| | CTKVT MDDFL TAHHE MGHIQ | | |
| | YDMAY AAQPF LLRNG ANEGF | | |
| S [5] | SYGFQPTNGVGYQPY | SARS- | SEQ. |
| | | CoV-2 | 20 |
| | | s.pro- | |
| | | tein | |
| | | RBD | |
| EK1 | SLDQINVTFLDLEYEMKKLEEAIKKL | 2019- | SEQ. |
| [6] | EESYIDLKEL | nCoV | 21 |
| | | S-HR1 | |
| | | (heptad | |
| | | repeat | |
| | | 1) | |
| HR1-1 | NGIGVTQNVLYENQKQIANQFNKAIS | SARS- | SEQ. |
| [7] | QIQESLTTTSTA | CoV | 22 |
| | | HR1 | |
| | | (heptad | |
| | | region) | |
| SARSww- | MWKTPTLKYFGGFNFSQIL | SARS- | SEQ. |
| 1 [8] | | CoV | 23 |
| | | spike | |
| | | protein | |
| Inhib- | SLDQINVTFLDEYEMKKLEEAIKK | SARS- | SEQ. |
| itor | LEESYIDLKEL | Co-V-2 | 24 |
| EK1 | *Bolded AA's represent | spike | |
| [9] | sites where salt-bridge | protein | |
| | formation occurs | | |
| Inhib- | SLDQINVTFLDEYEMKKLEEAIKK | SARS- | SEQ. |
| itor | LEESYIDLKEL-PEG4-C(PALM) | Co-V-2 | 25 |
| EK1P | *Bolded AA's represent | spike | |
| [9] | sites where salt-bridge | protein | |
| | formation occurs | | |
| | *PALM: palmitic acid | | |
| EK1C | SLDQINVTFLDEYEMKKLEEAIKK | SARS- | SEQ. |
| [9] | LEESYIDLKEL-PEG4-C(CHOL) | Co-V-2 | 26 |
| | *Bolded AA's represent | spike | |
| | sites where salt-bridge | protein | |
| | formation occurs | | |
| | *CHOL: cholesterol | | |

The strategy of developing small molecule, antibody, and peptide-based treatments targeting coronavirus spike proteins and receptor-binding domains (RBDs) has its roots in the body's native antibody defense mechanisms. The cryo-EM structure of SARS-CoV-2's ACE-2 binding spike protein was recently published, enabling researchers to more effectively rationally design and test drugs. This information facilitated the identification of a 23 amino acid peptide (SBP-1′: IEEQAKTFLDKFNHEAEDLFYQS [SEQ. 27]) within human ACE-2 which binds SARS-CoV-2. Molecular dynamic simulations revealed that spike binding protein-1 SBP1′ [SEQ. 27] binds with μM affinity to the SARS-CoV-2-RBD, strongly suggesting a peptide-based drug can inhibit SARS-CoV-2 spike protein binding with human lung epithelial ACE-2, preventing infection. Site-specific mutation was used to optimize the SBP1′ [SEQ. 27] sequence and generate SBP2 [SEQ. 29], with improved affinity (100's nM affinity) Table 3. Similar strategies have shown promise in the treatment of other viruses. The peptide-based drug Enfuvirtide®, for example, inhibits HIV viral entry by binding to the viral protein gp41; poor peptide stability, however, necessitates twice daily injections and adjunctive HAART to help maintain a low viral load. In addition to inhibition of viral binding, it is within the scope of this invention to create a ‘functional opsonin’ that mimics the body's natural antibodies mechanism, through tagging virions for degradation. Recombinant hACE2 and virus-specific antibodies from convalescent patient plasma have been explored for this purpose. However, these techniques are inferior to peptide-based strategies because of the risk of blood-borne disease, limited availability, and high cost.

The present invention overcomes challenges presented by other therapeutic strategies. As with Enfuvirtide®, poor peptide stability is a major concern for a potential peptide-based COVID-19 therapeutic in vivo. The lack of non-covalent interactions leaves most peptides exposed to rapid diffusion, proteolysis and degradation within minutes to hours, whether administered orally, intravenously or even subcutaneously. Self-assembling peptides (SAPs) circumvent this issue through non-covalent stabilization and supramolecular interactions. An SAP platform was developed by the present inventors that spontaneously forms β-sheet based nanofibers upon dissolution into an aqueous solution, thereby stabilizing therapeutic peptides. The invention will yield a scalable targeted treatment for COVID-19, which can be deployed both as a prophylactic to decrease infection risk and as a treatment option for those already infected by the virus.

TABLE 3
Additional Peptide Sequences for Binding
to Antigenic Sequences and related Plaque
Reduction

| | | Plaque | |
|---|---|---|---|
| | | Reduc- | |
| | | tion | |
| | | (10 μM, | SEQ. |
| Name | Amino Acid Sequence | n-4) | NO. |
| SBP1' | IEEQAKTFLDKFNHEAEDLFYQS | 15% | SEQ. |
| | | | 27 |
| ESBP1 | ESLSLSLSLSLSLE-G- | 84% | SEQ. |
| | IEEQAKTFLDKFNHEAEDLFYQS | | 28 |
| SBP2 | SALEEQLKTFLDKFMHELEDLLYQLAL | 88% | SEQ. |
| | | | 29 |
| ESBP2 | ESLSLSLSLSLSLE-G- SALEE-G- | 74% | SEQ. |
| | QLKTFLDKFMHELEDLLYQLAL | | 30 |
| KSBP2 | KSLSLSLSLSLSLK-G- SALEE-G- | — | SEQ. |
| | QLKTFLDKFMHELEDLLYQLAL | | 31 |
| SBP3 | QYKTYIDKNNHYAEDERYK | — | SEQ. |
| | | | 32 |
| ESBP3 | ESLSLSLSLSLSLE-G- | — | SEQ. |
| | QYKTYIDKNNHYAEDERYK | | 33 |
| KSBP3 | KSLSLSLSLSLSLK-G- | — | SEQ. |
| | QYKTYIDKNNHYAEDERYK | | 34 |

### Experimental Section

Preliminary Data:

Again, it is hypothesized that SAPs can be functionalized to bind to the SARS-CoV-2 spike protein's ACE-2 RBD, preventing fusion, and self-assembling into nanofibers on the viral surface, potentially promoting opsonization. The present investigator has demonstrated several key features of the SAP platform that support the present hypothesis and lend themselves to the development of opsonin-like antiviral therapeutics:

(i) SAPs are customizable solid phase peptide synthesized peptides. They are based on the canonical sequence K-(SL)₆-K [SEQ.1] which aggregates via β-sheet interactions of the (SL) midblock to form fibers with polar lysine termini.^{47,51} These 10-15 nm wide, 1-2 nm tall, 100s nm-μm long nanofibers are highly soluble >40 mg/mL (4w %) in aqueous phosphate buffer, FIG. 2 A-D.^{57,58,60}

(ii) The non-covalent supramolecular assembly of SAPs allows shear thinning and recovery (thixotropic behavior) of nanofibers yielding injectable, soluble self-assembled drugs (FIG. 2B).⁵⁸ Notably, self-assembly is potentiated by the presence of multivalent anions such as PO₄³, growth factors/cytokines, large multivalent charged drugs, and nucleic acids.^{48,50,51,61}

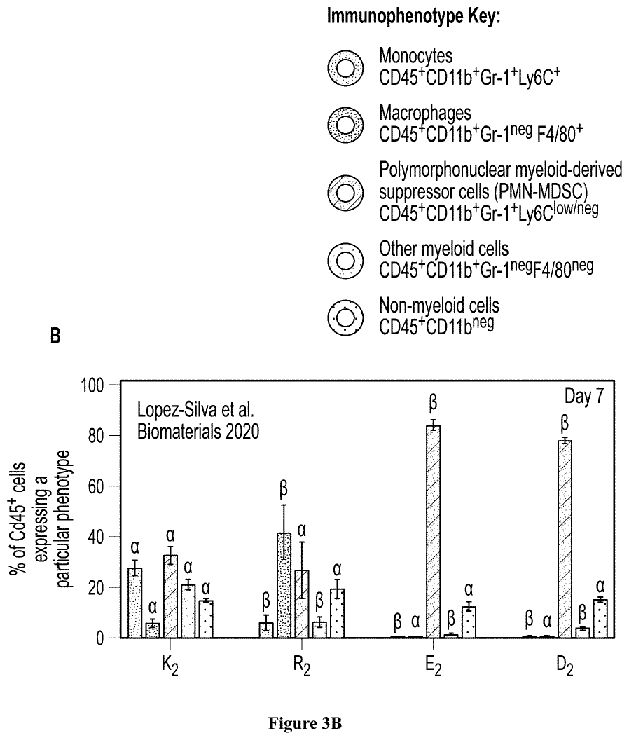

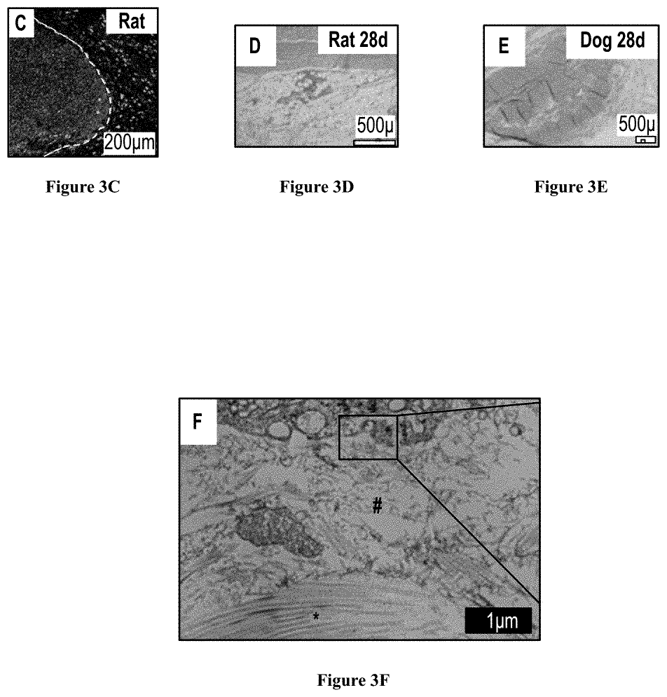

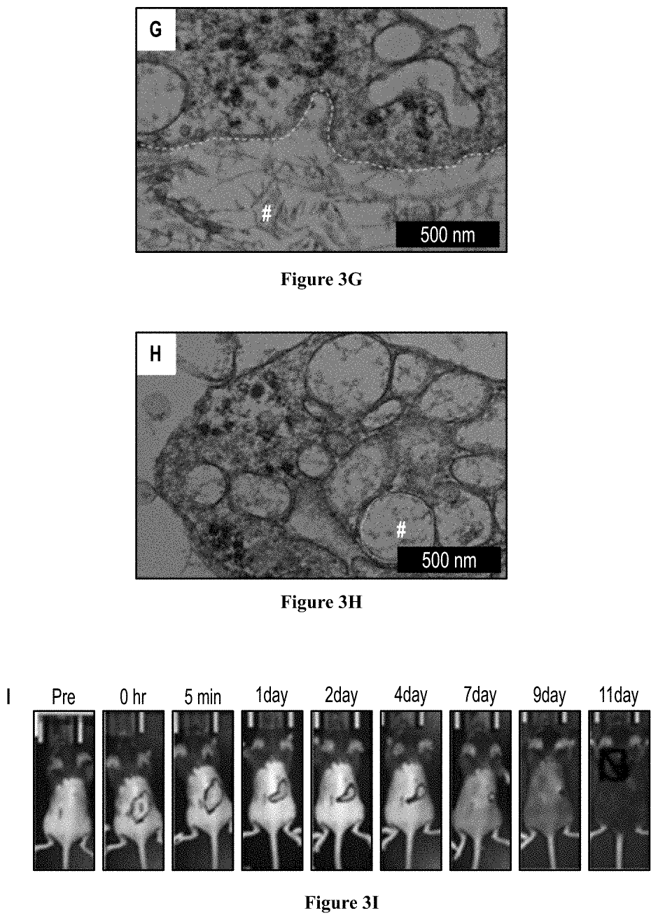

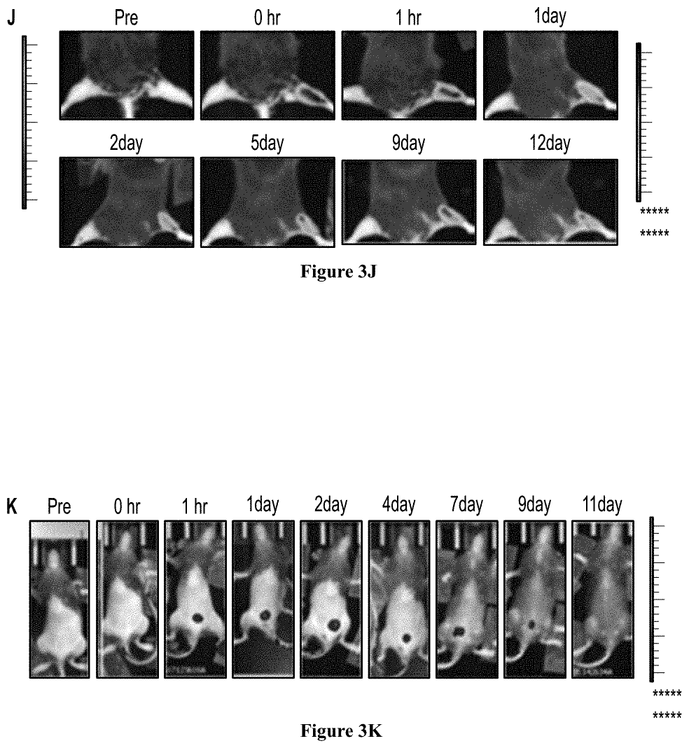

(iii) These ionic interactions allow enhanced stability and attenuated release of otherwise short half-life drugs or other molecules⁵⁰; and can be exploited for lyophilized shelf storage and stabilization (FIG. 2C). Notably, in vivo subcutaneous boluses of SAP nanofibers show rapid infiltration of cells (within 7 days to the center of 200 μL implants in rats or mice, FIGS. 3A-3K, showing easily retrievable boluses).⁴⁸ β-sheet self-assembly allows optimization of nanofiber aggregate degradation over a 1 month period compared to 3-7 days for peptide amphiphiles or similar β-sheet forming hydrogel Puramatrix, FIGS. 3A-3K^{48,50,51,58,62} Complementing the CD68+, macrophages within implants are host to the innate immune system depending on tunable peptide functionality allowing tweaking of the platform to achieve the goals of this proposal, FIGS. 3B-3E.^{55,66}

(iv) A robust localized immune response is noted only towards SAP nanofibers with minimal inflammation in the surrounding fascia of Sub-Q or IM implants—demonstrating excellent biocompatibility, FIGS. 3A-3K.^{47,51,61,66}

(v) Antigen presenting cells endocytose peptide fibers into vacuoles as noted in TEM studies, FIGS. 3F-3H. Furthermore, CD68+ macrophages that populate the implants (1×50 μL SC implants in mice, 6×200 μL in rats, and 16×1 mL in dogs) recruit other mesenchymal cells that deposit collagen matrix, leaving native tissue within 1 month of peptide implant degradation into native L-amino acids, FIGS. 3C-3H.^{47,51,61,66}

In addition, it is demonstrated by the present investigator that the facile addition of short peptide mimic (10-30 amino acid) sequences of biological molecules derived from phage display or computational analysis onto the base K-(SL)₆-K [SEQ. 1] sequence: K-(SL)₆-K-G-[SEQ.12] “mimic”.^{51,55,57-60,64,67} These synthetic peptides assemble into nanofibers that present the mimic epitope in high local density.^{47,51,55,58,67} Using this technique, the present investigator has developed and published angiogenic, neurogenic, cytokine binding, dentinogenic, anti-microbial, protein-binding (PCSK-9) (FIG. 2F) and anti-angiogenic scaffolds that have localized in vivo effect.^{48-51,55-61,65,67} Importantly, surface plasmon resonance has shown similar binding kinetics for mimic functionalized SAP—but very slow detachment compared to the mimic alone, (FIG. 2F).

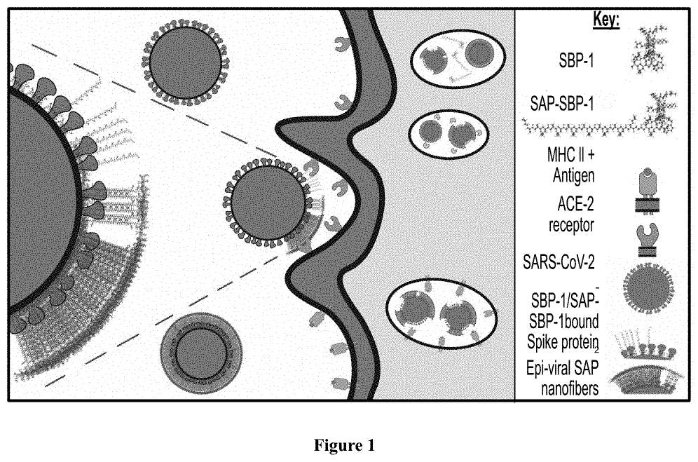

(vi) Functionalized peptides bind their cognate receptors and self-assemble into fibers—potentially opsonizing targets for macrophage endocytosis. The present investigator has shown the addition of a variety of peptide mimics and their binding to targets demonstrated by SPR (FIG. 1F), ELISA, FACS, in vitro cell proliferation, in vitro cell differentiation, in vivo cell and recruitment and differentiation.^{48,50,51,55,58,61,67}

The overall strategy is a rationalized approach to address the hypotheses presented. In terms of literature weakness, it has been identified the rationale, stable small molecule therapeutic approaches and a clinically analogous small animal model and key missing areas. The investigator reviewed the prior literature and determined the presented studies described herein are required for a robust and unbiased development of a therapeutic and small animal screen model.

Specific Rationale: The studies herein investigates the potential mechanism of a novel drug and its interaction with SARS-CoV-2 to reduce viral burden. Also investigated is the ability to store the peptide-drug in lyophilized form on the shelf for months to years, and manufacture at low cost—allowing for large scale utilization. It will simultaneously be evaluated that the in vitro binding using to SARS-CoV-2 RBD, in vitro efficacy using Caco-2 cell-based SARS-COV-2 infection and TEM imaging of epi-viral self-assembly.

Overview of the Approach: Again, it will simultaneously be evaluated that the in vitro efficacy and in vivo maximum tolerable dose is tested using a developing golden Syrian Hamster model in the Co-PI's lab, and TEM imaging of epi-viral self-assembly. Simultaneous execution of both independent aims in parallel is to rapidly establish efficacy and a suitable clinical dosing can be evinced.

Choice of Peptide Inhibitor: The rapid publication and open access nature of COVID-19 literature has allowed to perform a thorough review of the relevant literature to determine alternative peptide inhibitors of COVID-19.^{1,5,6,14,28,72-74} These ongoing literature reviews will continually inform the design of the anti-COVID opsonin. Notwithstanding, the approach proposed is facilely adapted platform for a growing list of peptide inhibitor sequences (https://bit.ly/2VFPLFe) (not reproduced for space considerations). As the literature grows, the present disclosure can be judiciously adapted if a superior binding partner is discovered.

Aim 1: Characterization of a functional peptide that binds ACE-2 and in vitro efficacy. Synthesis and stability of novel peptides is optimized to target low cost (<$1/dose) and 1 g-100 g scale of peptide material of over 90% purity. Binding kinetics determined using BLI or SPR of candidate peptides is compared to SBP1 to optimize peptide design and eliminate poor binding candidates. Negatively stained TEM will be used to visualize peptide virion binding and potential epi-viral fibrillation/opsonization. Below are experiments that are representative of achieving these objectives. The experiments listed below are merely representative for illustration purposes and are not meant limit the possible variations utilizing the teachings herein.

Experiment 1: Synthesis and characterization of peptides: Synthesized is the midblock (SL)₆ self-assembling domain flanked by K2, R2, D2 or E2 termini, with a glycine spacer, and the 23 amino acid ACE-2 RBD (SBP1). This will yield 4 peptides of sequence: K2-SBP1, D2-SBP1, R2-SBP1 or E2-SBP1 (and their respective controls—SBP1, a scrambled SBP1sc, K2, R2, D2 and E2), See Table 1. Peptides will be formulated in DI water and 10×PBS (9:1).^{51,58,59,61} Developed is a LC/UV method to detect the purity of 3 individual 0.1 mM scale synthesis (approximately 100-1000 single doses), and use LC/MS to determine any impurity >1% by weight. Further, manufactured is an isotope labeled standards (New England Peptides) and initiate storage stability of lyophilized peptide at −20° C. for 12 months, and 40° C. accelerated stability in a 65% humified chamber (to simulate shelf stability for 2 years)—(n=8) weekly; with LC/UV to determine a degradation profile.

Experiment 2: In vitro receptor binding kinetics: Surface plasmon resonance is used to verify binding: His-tagged 2019-nCoV S is immobilized to an NiNTA sensor chip in a Biacore X100 using HEPES buffer and surfactant. Serial dilutions of test compounds in table 3 (500 to 1 nM) is compared to recombinant ACE2 and SBP1 as known binding controls.^{25,52} The resulting data from SPR association and dissociation will help determine how SAP forms a strong adhesion (with poor dissociation kinetics) to SARS-CoV-2 indicating opsonization potential (n=6 based on expected error and effect size).^{1,25,52-54}

Experiment 3: Negative stained TEM to visualize opsonization—epi-viral nanofiber self-assembly: Purified 2019-nCoV S and whole virus is mixed with soluble recombinant human ACE-2, SBP1, or SAP-SBP1 at a range of concentrations determined by SPR. The mixture is fixed and deposited on a Cu grid stained with methylamine tungstate, and imaged using transmission electron microscopy (TEM).^{25,52} While SBP1 peptide-virus interactions may be difficult to ascertain with high resolution; fiber-virus interactions (indicative of opsonization) can be more easily visualized; fiber structural analysis at different concentrations in TEM is correlated to sizes in dynamic light scattering (DLS), TEM and AFM as above.

Experiment 4: Inhibition of Virus-Induced Cytopathogenic Effect: A well-established in vitro infection model will be utilized to test.^{15,75} Human colon carcinoma cell line (Caco-2) cells are cultured in Minimal Essential Medium (MEM)+10% FBS and 1% pen-strep. SARS-CoV-2 will be isolated from human samples and cultured in Caco-2 cells. Confluent layers of Caco-2 cells in 96-well multi-well plates (50,000 cells/well) are challenged with SARS-CoV-2 at a multiplicity of infection (MOI) of 0.01. The virus is added with the SAP(s) under investigation and incubated in MEM supplemented with 1% FBS. Remdesivir® and SBP-1 are positive controls, and no treatment/scrambled SBP-1 is used as a negative control. Three independent experiments of n=4 per group are performed at 0.1 nM, 1 nM, 10 nM, 100 nm, 1 μM concentrations to determine optimal formulations and range of concentrations. Cells will then be cultured for 48 h with subsequent cytopathogenic effect (CPE) visually scored by two independent investigators and verified using MTT. A dose-response curve is generated and the EC50 (inhibition of 50% of infections) calculated based on the CPE.^{15,75}

Experiment 5: Assessment of Cell Viability: The viability of Caco-2 cells treated with varying concentrations of functionalized SAP (as above) are assessed using Rotitest® cytocompatibility for 3 independent experiments of n=4/group. The CC50 (cytotoxic concentration of SAP resulting in cell viability decrease by 50%) is calculated from a dose-response curve modeled using four-parameter curve-fitting. Cell studies in 2.1 and 2.2 are conducted in Co-I: Subbian's lab at Rutgers.

Concerning the success metrics, optimization of the current synthetic scheme is to bring cost <$1/10 mg dose, and >90%+ purity—sufficient for peptide drug translation.^{15,43} It is also to determine and optimize storage conditions for <10% degradation over 1 year—utilizing accelerated testing methodology.^{45,46} Furthermore, it is measured by evaluation and superior binding (and slower dissociation) of novel peptides compared to SBP1 alone.^{1,52,53} Also obtained is a potential image and determination of a mechanism for peptide-binding and self-assembly on the surface of virions.^{52,53} Metrics also can determine the ability of SBP1 and SAP analogs of SBP1 to prevent binding of COVID-19 to ACE-2 on Caco-2 cells in a dose dependent fashion, compared to clinically utilized fusion/entry inhibitor Remdesivir®. The metrics can establish an EC50 and CC 50 based on the potential cytotoxicity of high concentrations of peptide that has not been seen in prior studies—surprisingly high concentrations of SAP result in hydrogelation and creation of a matrix for mammalian cell proliferation; and establish a selectivity ratio to carry no more than 2 candidates into in vivo safety studies.

The potential pitfalls and alternative strategies for the above include, but are not limited to the following. It may be noticed that E2-SBP1 or K2-SBP1 may not bind with higher affinity to the RBD, compared to SBP1. To this end, it may be desirable to optimize the termini for other charged residues, increase the glycine spacer length (1,3,5 glycine), increase the number of (SL) repeats in the midblock to promote fiber stability, reduce steric hindrance, and allow incorporation of longer linkers such as PEG4 or related linkers.

Alternative peptide/polymeric strategies can also be envisioned given the biomaterial and drug delivery expertise of the PI. Given the rapidly growing literature on COVID-19 and inhibitory molecules—an infrastructure can be built to rapidly evaluate other peptide candidates for efficacy plus or minus the present SAP platform. The SAP conjugated variants may not show expected super-stoichiometric binding like previous studies, FIG. 1. Other binding domains or peptides (detailed in Aim 1) can be attempted.^{1,52,53,76} Self-assembling peptides with the SBP1 domain may, unexpectedly, become cytotoxic (unlike the over dozen other constructs evaluated). However, if this occurs, alternative peptide sequences which promote cytocompatibility, for example—RGDS, may be added to promote cytocompatibility.^{47,48} Alternatively, other peptide/peptide amphiphile approaches can be utilized.⁷⁷⁻⁸⁰ Possible exploration of a toxicity study in canines to ensure 2 species safety (and confirmation of dosing range) is possible prior to first in human studies.

Aim 2: Evaluation of a small animal in vivo efficacy model to screen therapeutic candidates Anti-viral compounds, which were found effective against SARS and MERS, could be repurposed to treat SARS-CoV-2 infection. The nucleoside analog, Remdesivir® showed EC₅₀=0.77 μM against SARS-CoV-2 in vero E6 cell line.⁸¹ An anti-malarial drug, Chloroquine is reported to have broad-spectrum anti-viral activity by inhibiting the glycosylation of cell receptors for SARS-CoV and immunomodulation. The pneumonia and acute respiratory distress syndrome is caused by cytokine storm during COVID-19.^{11,82,83} Selective cytokine blocking during COVID-19, using either IL-6 blocker (tocilizumab) was (approved in China for patients with increased IL-6 during COVID19) and IL-1 blocker (anakinra) might be beneficial in reducing hyperinflammation.^{11,84,85} And JAK-inhibitor baricitinib was found to inhibit virus entry as well as hyperinflammation⁸⁶ and it needs to be evaluated (Richardson, 2020). In the present study, investigated is the anti-viral activity of Remdesivir®, IL6 blockers and the SAP optimized in Aim 1.

Mice, non-human primates (NHP), hamsters and ferrets have been investigated for being a model for SARS-CoV, a member of beta coronavirus related to SARS-CoV-2. Mice show poor virus replication and pathological evidences,⁸⁷ NHP and ferrets supported virus replication and pneumonitis for short durations⁸⁸ and mild pathological lesions^{86,89,90}, respectively. However, hamsters showed high titer virus replication, viremia, pneumonitis and extrapulmonary spread to liver and spleen for the duration of 7 to 10 dpi before virus clearance.⁸⁷ On in silico analysis, the spike protein RBD domain of SARS-CoV-2 was found interact with hamster ACE2 in high affinity⁹¹ Chan and co-workers reported that on SARS-CoV-2 infection, golden Syrian hamsters had clinical signs, virus replication, histopathological changes mimicking human COVID-19, so, in the present disclosure it will be using golden Syrian hamsters in the current study to investigate virus-host responses, and the therapeutic efficacy of anti-viral agents.⁹¹

Experiment 1 To understand the viral infectious dose (ID) response in the hamster model the following is proposed. Obtained are 8 weeks old golden Syrian hamsters (source) and will cage them as 8 hamsters (4 males and 4 females) in each group. Obtained is SARS-CoV-2 from the Rutgers Coronavirus Research Consortium. The virus will be amplified in Vero E6 cell line by passaging once to prepare working virus stock^{91,92} and confirmed by qRT-PCR^{91,93}, and virus infectivity assays (TCID₅₀).⁹¹ Each group of hamsters is infected through intranasal route with various infectious units of virus-10^{2.5}, 10⁵, 10^{7.5} plaque forming units of virus (pfu). Hamsters will be monitored for clinical manifestation of disease and physical activity (using activity wheel) and recorded periodically. Animals will be euthanized at 2, 4, 7, and 14 dpi⁹¹ and samples including blood, nasal wash/nasal swabs, nasal turbinate, trachea, lungs, lymph nodes, heart, liver, kidney, intestine and feces will be collected in appropriate media. Viral load will be determined from collected samples using qRT-PCR, and virus infectious titer will be determined by infectivity assays in Vero E6 cell line.⁹¹ RNA-seq data⁹¹ will be generated, histopathological examinations and IHC will be performed on above samples.⁹¹ Blood parameters will be quantified and the cytokines/chemokines involved in cytokine storm during COVID-19 pneumonia such as IL-1, IL-6, TNFa, CCL2, IFNg, GCSF, IP-10, MIP-1a will be assayed from blood and lung tissues by multiplex Luminex kit and qRT-PCR.⁹¹ The differential expression of cytokines is correlated with, viral load and histopathological changes with various infectious virus dose.

Experiment 2 Determining the therapeutic efficacy of Remdesivir®, inflammatory IL-6 mediators and SAP peptides in golden Syrian hamster models is proposed. Based on the viral load and histopathological results obtained in Experiment 1, the determination of appropriate time point post infection is done to initiate treatment with the following compounds; Remdesivir®. SAP candidates (0.01, 0.1 and 1.0 mM) and the IL6 blocker Tocilizumab.

Notably SAP doses are well below (<˜10×) what has been attempted in prior reports of SAP, and significantly higher than the nM-μM which will be used to study binding kinetics in Aim 1. Appropriate control groups will include: untreated, virus infected/untreated and virus infected treatment groups—7 groups of 8 (4M/4F) hamsters. These compounds will be administered to animals by IV daily to maintain the plasma levels. During the treatment, monitoring will continue of the animals, collection of blood and sacrifice at the time of treatment, 2, 4, 7, 14 days of treatment. Organs including blood will be collected as well as nasal wash, lungs, lymph nodes, nasal turbinate, trachea, heart, liver, kidney, spleen, feces. Measured are viral load and quantify the infectious titer the from blood, lungs and nasal turbinate by qRT-PCR and viral infectivity assay (TCID₅₀),⁹¹ respectively as described in Experiment 1 above.

Histopathological analysis on organs, and cytokines will be assayed from blood and lungs as described in Experiment 1. Assessment is done of the toxicity of the drugs by measuring liver enzymes, cardiac enzymes, and kidney function tests and histopathological examination of liver, kidney, and heart. The success metrics and viral load will be assessed as follows. Observation of clinical features in SARS-CoV-2-challenged animals including weight loss, lethargy, fur quality, posture, and breathing rate. Determination of viral load in upper and lower respiratory tract, extrapulmonary organs, and blood from virus-challenged animals at different time points (2, 4, 7, and 14 dpi) to see if nasal turbinate, lung, intestines, and trachea tissues had highest viral load as compared to extrapulmonary tissues. Furthermore, determination of viral titer in previous tissues to see if lung tissue had highest value to similar ensure airway involvement of SARS-CoV-2 in hamsters as compared to humans. Observation and imaging of epithelium in upper and lower respiratory tracts and extrapulmonary organs to determine whether there is intra-epithelial and submucosal infiltration, viral N protein expression, cell swelling, infiltration of lymphocytes/monocytes. Also, determination of cytokine/chemokine levels in lungs of virus-infected animals at previous timepoints and see if it is comparable to cytokine storm observed in COVID-19 in humans. From this data, an appropriate timepoint to introduce treatment based on high levels of viral load and cytokine/chemokines will be determined in order to begin Experiment 2. Therapeutics: i) verification of viral inhibition using Remdesivir® with comparison to human dosing; ii) mitigation of pro-inflammatory cytokines using IL-6 inhibitors; iii) (optimizing dosing for) eradication of virus from titers using SAP. Specific to the latter—it will be determined for the maximum tolerable/NOAEL of peptides and SAP modified peptides along with a PK profile determined by LC/MS.

Potential pitfalls and alternative strategies for Aim 2 include the following of the Viral load. There may have to be higher efforts into finding data used to compare viral load, titer, and cytokine/chemokine levels to those observed in humans. However, the open access of COVID-19 related literature may allow access to similar data using human subjects, and help determine if the golden Syrian hamster is a similar enough host that studies using it as a subject will be adequate. If any evidence of toxicity is seen in the animals after administration of any of the above compounds, a lower dose will be considered for an additional treatment group given the nM affinities seen in prior SPR data and the safety of the present established mM IV dosing of SAP. This allows for higher doses in the event no mitigation of viral load and infectious viral titer is observed.

To ensure scientific rigor, followed is a Data Sharing Plan to ensure citation and publication of novel or altered methods. The plan is to address weaknesses in the rigor of prior research. Current non-specific and experimental treatments for COVID-19 are rationalized by the immediacy of the pandemic. Proposed herein is a rationalized approach that can only now be attempted given the recent elucidation of the spike protein crystal structure.^{1,52-54} A key oversight of previous research studies^{48,51,68-70} is the lack of separation of the data collection from data analysis. In the present workflow, SAP-specific data collected from academic collaborators' labs will be “anonymized” as to the identity of the SAP or control and assigned a code. The data will then be analyzed in the principal investigator's (PI's) lab. After analysis, the SAP or control will be de-anonymized and reported. This workflow will be reversed for the data generated in PI's lab and note that differences in sexes may be observed. In the event differences are seen in the present animal cohort—it will disambiguate the data (potentially request for more animals depending on error and significance) and present the differences as a function of sex or other variables as appropriate. Note on sample sizes and statistical analyses: A group size of 5-6 (unless otherwise noted) is large enough to detect a 50% difference in mean values at a power of 0.8 and p<0.05 based upon standard deviations obtained in prior studies (10%).^{1,6,51,52,55,95} Comparisons will be made using Student's t-test for paired data and analysis of variance (ANOVA) for multiple comparisons with Tukey post hoc analysis for parametric data and Dunn's post hoc analysis for non-parametric data.^{48,51,55,96}

### Discussion of Results

Adverting again to the Figures, shown is FIG. 1 a schematic of the proposed self-assembling peptide opsonin strategy. The ACE-2 binding domain peptide sequence (SBP1) of a targeted self-assembling peptide (SAP-SBP1) can bind SARS-CoV-2 viral particles, preventing them from binding human lung ACE-2 receptors. Fibrillization of peptides on the viral surface may target the virion for endocytosis and proteolytic cleavage (functional opsonization). Antigen presenting cells may then process and present SARS-CoV-2 antigens, leading to immunity.

FIGS. 2A-2F are photographs and graphs showing an embodiment of the design of the self-assembling peptide (SAP). FIG. 2A are SAPs designed to have a base fibrillizing domain (K-SLSLSLSLSLSL-K) [SEQ. 1] and customized bioactive domain, including but not limited to SBP1. In aqueous solutions, the hydrophobic pendant groups of the fibrillizing domain self-assemble into dimers, excluding water. The hydrophilic pendant groups of the serines promote solubilization by hydrogen bonding. FIG. 2E is prior known data by other investigators as used for illustrative purposes only.

FIG. 2B illustrates peptides that further self-assemble into tetramers, oligomers and nanofibers. FIG. 2C shows that Synthesized peptides can be lyophilized into a stable white powder for solubilization into a viscous solution immediately upon complete dissolution. FIG. 2D is an Atomic Force Microscopy (AFM) picture that shows the formation of 10-15 nm wide 1-2 nm thick ribbon-like nanofibers of 100s nm-μm length that entangle and solvate to create nanofibers at up to >4w % in phosphate buffer.

FIG. 2E is a chart showing the Pentelute group has shown SBP1 to bind to the SARS-CoV-2 RBD at nM (10-50 nM) concentrations using biolayer interferometry. FIG. 2F is a chart showing the Surface Plason Resonance (SPR) analysis of a PCSK9 binding SAPH shows that peptide mimics can bind dose-dependently and specifically to their cognate receptors over 60s of binding, and dissociate rapidly from the chip (left inset). SAPH peptide's mimic binds to the same cognate receptor, irreversibly and at a high binding affinity (y-axis), suggesting stronger binding, slower release and potentially self-assembly atop receptors. This results in super-stoichiometric binding of peptides to receptors vs. no appreciable binding to a blank carbonic anhydrase chip (right inset). Conjugation of SBP1 to the fibrillizing backbone may create fibers that bind in a super-stoichiometric fashion to target proteins.

FIGS. 3A-3H are photomicrographs and charts showing biocompatibility and intracellular trafficking and are for background and illustrative purposes only. SAP are injected via 27-30 g needle under the dorsal skin of mice, rats and dogs at multiple sites. One of the advantages of the present invention is that it maybe injectable. FIG. 3A illustrates high concentrations of peptide implants that can be retrieved at early timepoints (3, 7 and 14 days) before complete biodegradation and processed for Fluorescence-activated cell sorting (FAC) analysis. FIG. 3B shows FACS analysis that K-terminated peptides have suitable APC repopulation at 8 days with lesser involvement of PMN neutrophils (Lopez-Silva et al). FIG. 3C illustrates that boluses can be blocked and immune-stained to verify macrophage cellular infiltrate (CD68+ red, CD163+ purple) primarily within the implant (left of dotted line), with minimal effect on the surrounding fascia (right of line) or systemic effect at 7 days in rats.

FIG. 3D and FIG. 3E are photomicrographs shown as 200 μL boluses of peptide Sub-Q in rats and dogs, respectively, degrade within 28 days, while 1 mL boluses in canines can still be easily visualized biodegrading, with a highly localized response—indicating biocompatibility in canines, rats and mice. TEM images of the cells that infiltrated the center of scaffolds in mice at 7 days showed invaginations of endocytosed peptide, shown in FIG. 3F and magnified in FIG. 3G, present in intracellular vacuoles as shown in FIG. 3H.

Peptide fibers are noted (#) in proximity with collagen (*) identified by their canonical D-periodicity in the center of the implants. Lack of fibrous encapsulation, excellent implant infiltration and host-implant integration, deposition of neo-matrix verified by TEM and trichrome all indicate biocompatibility and a highly local immune response in a number of animal species. Note that in FIG. 3I and FIG. 3J the 50 μL Sub-Q and 25 μL IM boluses in 25 g C57BL/6 mice self-assembles into nanofibers in situ after injection and degrade over weeks. In FIG. 3K the thixotropic SAP flows with hemodynamic shear to the bladder and is excreted over time.

As previously discussed, in one embodiment a rationalized approach (peptide design using BUDE alanine scanning, SWISS-Model, Rosetta, and binding stability using molecular dynamics (MD)) facilitated the development of peptides with novel pan-coronavirus functionality, and will address the current (SARS-CoV-2), previous (SARS), other coronaviruses (i.e., MERS, HCoV-229E, HCoV-NL63, HCoV-OC43, and HCoV-HKU1); and future pandemics. Critically, explored herein is the ability of the SAP domain to guide assembly atop viral particles (visualized with Co-I Prasad), potentially clustering atop antigenic determinants, and allow for the opsonization of viral targets (see Preliminary data below). Development of a targeted pan-coronavirus binding opsonin utilizes concepts of the present invention. Through computational design, developed was a (19-mer) peptide, SBP3, that can bind specifically to SARS-CoV-2 and other SARS, in silico. Facile modification with the present inventor's self-assembling peptide (SAP) platform results in a construct (ESBP3) that allows 10 nM IC50 inhibition of live-virus infection of Vero cells in vitro, with a robust rodent in vivo IV safety profile (at mM daily dosing for 10 days). Again concepts of the invention utilize the following hypothesis: 1) a functionalized self-assembling peptide (SAP) can bind the SARS-CoV-2-ACE2 receptor binding domain of spike protein, thereby preventing infection, and can self-assemble atop the virion, thereby promoting opsonization; and 2) facile tuning of the peptide sequence through rationalized computational design can allow pan-coronavirus targeting. A new platform for the treatment of viruses is proposed by the concepts of the current invention to generate and characterize an SAP which first specifically targets virions and next promotes endocytosis by functioning as an opsonin. Binding kinetics of the SARS-CoV-2-RBD with ESBP3 peptides, and characterize subsequent SAP self-assembly are characterized as shown in, FIG. 2A-2D. This self-assembly of opsonin-like materials on the virion surface, potentially targeting the virion for immune destruction, is a unique approach that goes beyond the use of decoy receptors or mimicking proteins (SBP1 alone) to block binding. It is possible that our strategy will lead to the development of immunity through the phagocytic clearance in vacuoles, resulting in a potential presentation on MHC II. Furthermore, sequence homology among coronaviruses (e.g. the 9-20 nucleotide difference between SARS and SARS-CoV-2) suggests that the drug identified herein may have potential broad neutralizing ability. This novel treatment strategy (epi-viral self-assembly, designer opsonin development, and the use of SAPs to achieve these effects) may be applicable to other infectious diseases.

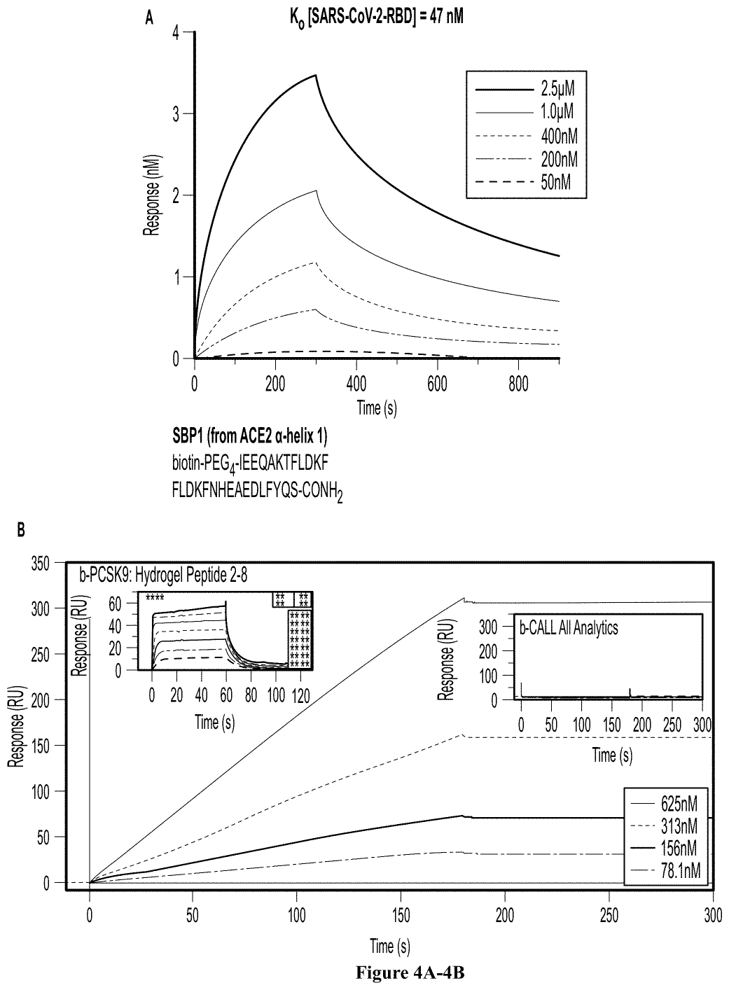

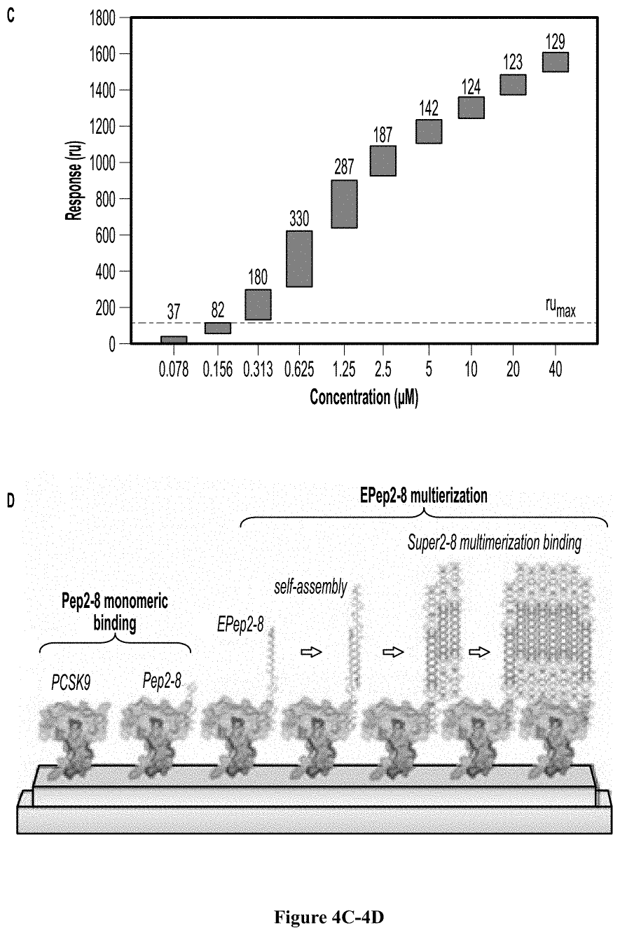

The present invention establishes a novel mechanism of underpinning therapeutics opsonins. Given the promising nM efficacy of the present invention's designer SAP against the live virus, compared to non-SAP versions, the ability to determine a unique mechanism of action governing SAP efficacy is crucial. With the qualified production of Spike protein and related mutants by several vendors, SPR can be used to study further the super-stoichiometric binding observed in Preliminary data, as well as for other peptides developed within this platform, As shown in FIGS. 4A-4D, advances in cryo-EM imaging may also allow the visualization of live virus “coated” and opsonized through association with SAP. Further, the computational design of pan-coronavirus targeting constructs will complement the EM and SPR to rapidly design and deploy pan-coronavirus therapeutics.

FIGS. 4A-4D illustrate the super stoichiometric binding concept utilized in the present invention. FIG. 4A shows that SBP-1 binds at μM concentrations to SAR-CoV-2 RBD using biolayer interferometry. FIG. 4B illustrates SPR of a functinoalized SAP binding PCSK9 and shows that peptide mimics can bind dose-dependently at a high binding affinity (y-axis) and irreversibly—suggesting stronger binding, slower release and potentially self-assembly atop receptors, versus no appreciable binding to a blank carbonic anhydrase chip (B—right inset) or rapid dissociation of functional non-SAP peptides rapidly from the chip (B—left inset). FIG. 4C illustrates the functionalized SAP accumulation on target ligand bound surfaces. Values above each bar represents the maximum ru binding achieved during the injection. The dotted line, ruₘₐₓ, indicates the theoretical maximum binding for a 1:1 interaction (135 ru). FIG. 4D shows SAP may bind AND self-assemble on immobilized ligands, giving rise to super stoichiometric binding (>135 ru) and greater binding affinity as demonstrated for multiple functionalized SAP peptides. Conjugation of SBP mutants to the fibrilizing back bone, may create fibers that bind in a super-stoichiometric fashion to target proteins (potentially uncovering an opsonization strategy) for pan-coronavirus targeting.

Utilized in the current invention is the premise that SAPs can be functionalized to bind to the SARS-CoV-2 spike protein's ACE-2 RBD, preventing fusion. SAPs will self-assemble into nanofibers on the viral surface, potentially promoting opsonization. Several key factors that support these contentions include the following.

1) SAPs are customizable: The SAPs discussed in this application are efficiently produced using solid-phase peptide synthesized (SPPS) and are based on the canonical sequence E-(SL)₆-E shown in [SEQ.28], [SEQ. 30] which aggregates via β-sheet interactions of the (SL) midblock.

Formed are fibers with polar glutamic acid termini. These ˜10 nm wide×1-2 nm tall, 100s nm-μm long nanofibers are highly soluble >40 mg/mL (4w %) in aqueous phosphate buffer, FIG. 3. Demonstrated previously was 100 mg lab-scale and 1 g-scale (non-GLP, CMO) syntheses of these peptides within 24 hr. Given straightforward SPPS synthesis with excellent (>90% crude purity), scalable manufacture (worldwide) of this peptide is assured.

2) SAP can be easily modified for super-stoichiometric binding: From cholesterol modulating PCSK-9 binding peptides to angiogenic peptides for therapeutic revascularization, it has been shown that this modification and targeting strategy is valid for a broad range of biomedical applications. An interesting and clinically relevant phenomenon facilitated by this two-domain architecture is guided super-stoichiometric binding of functionalized SAPs to target proteins for opsonization. Traditional binding partners show binding to target proteins in a 1:1 stoichiometric fashion, followed by disassociation at a rate similar to the association rate. Conversely, functionalized SAPs bind to the ligand with high specificity (strong Kon) and then continue to self-assemble together atop the target protein, achieving super-stochiometric ratios between the peptide and the receptor. Unlike traditional binding agents, which “wash-off” or dissociate when the buffer is flowed atop sensors, functionalized SAPs maintain robust binding with negligible K_{off}.

The disassociation constant of the present invention is demonstrably lower than most antibodies, with the functionalized SAP not releasing from target proteins even after detergent washing, as shown in FIGS. 4A-4D. From a peptide-binding perspective, this is a novel and interesting phenomenon that warrants both in vitro/in vivo validation of effect and further in silico analysis. Postulated is that enhanced receptor signaling (and in the case of ESBP3)—enhanced viral inhibition—may be due to i) targeted binding; ii) self-assembly and iii) potential clustering (of the receptor binding domain, or Spike protein) that can be studied using cryo-EM and in silico.

3) SAP demonstrate facile delivery: SAP β-sheets associate due in part to the hydrophobic side chains of leucine residues in the midblock. These non-covalent (Van der Waal's) interactions facilitate self-assembly into nanofibers that are shear responsive. Upon needle force, these thixotropic liquids shear thin and flow, only to reassemble after relief of shear stress. In toxicity and pharmacokinetic studies, injection of these peptides intravenously, subcutaneously, or intranasally does not show any adverse thromboembolic effect, and clearance is observed over 12-24 hours when administered intravenously FIGS. 3A-3K.

4) SAP demonstrate efficacy and safety: SAPs have been delivered SubQ, IM, IV, IN (mice); SubQ, IV, intracranial, intradermal, intratooth (rats); SubQ, intratooth (dogs) with no adverse effect of trafficking into the circulation (in the case of IV delivery—clearance in 12-24 hours). Importantly, a repeat dose of ESBP family peptides in rats (daily at 10 mM, 1% blood volume) has no adverse effect or observable weight changes in animals. Furthermore, the SAP family of peptides have shown no known adaptive immune responses or even local immune reaction—as peptides nanofibers bind their targets, biodegrade in 2-4 weeks Sub-Q, and cause no toxicity when dosed daily at 1% blood volume for 10 days. Preliminary studies as shown in FIGS. 3A-3K visualize how SAPs are phagocytosed and degraded in intracellular vacuoles, and promoting opsonization without a cytokine concentration.

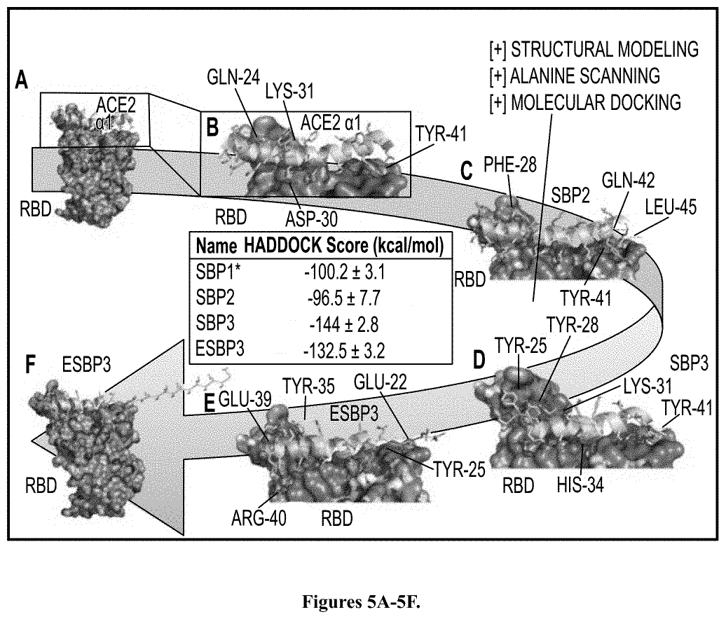

5) Computational design leverages the SAP platform for rapid response: Through SWISS-MODEL, alanine scanning mutagenesis, and HADDOCK molecular docking, developed was a short (19 residue) de novo sequence—SBP3—that binds to the Spike protein with μM-nM IC₅₀. Coupled with the SAP backbone, a 10 nM IC₅₀ was achieved. Complimentary Cryo-EM imaging has shown the formation of nanofibers of ESBP1/ESBP2/ESBP3 compared to aggregates/unassembled structures of binding domains alone. Further study of ESBP3 with live-virus (fixed) will help inform computational design and constraints derived through NMR and TEM. To elucidate a mechanism of epiviral binding, modeled was an ESBP3-Spike binding, ESBP3-Spike trimerization binding, and further used the present invention's computational platform for verification against other coronaviruses (as already demonstrated for SARS) as shown in FIGS. 5A-5F.

Shown in FIGS. 5A-5F are examples of workflow for Computational Modeling. FIG. 5A illustrates ACE2 α1 helix complexed on SARS-CoV-2 Spike RBD cropped from PDB ID: 6M0J. FIG. 5B illustrates key residues highlighted yellow deemed most significant on peptide and purple residues most significant on Spike RBD (as determined by ΔΔG). Derived from ACE2α1, SBP1 has been mutated for enhanced binding to RBD (termed SBP2) docked on Spike RBD via Haddock in FIG. 5C. FIG. 5D illustrates further mutation using structural modeling, alanine scanning and molecular docking, yielded SBP3 (with significantly improved Haddock scores over SBP1/2). FIG. 5E show interaction of ESBP3 monomer docked on Spike RBD, and the same zoomed out in FIG. 5F.

### Further Experimental Details

Aim 3: Mechanism—understanding SAP anti-viral drug action. Preliminary data show the ability of ESBP3 to bind and inhibit live-virus infection (USA-WA1/2020). This aim is to expand the utility of this strategy against B.1.1.7, B.1.351 and P.1 strain—demonstrating the facile and adaptable nature of this platform supporting in silico demonstration of inhibition efficacy. Sourced is (TOR2/wild-type strain) and tested for efficacy against SARS. A determination of the opsonization mechanism is done through SPR competitive binding analysis using SPR and Cryo-EM imaging (Co-PI Prasad). AFM and TEM analysis of neutralized viral titers preincubated with SAP opsonins will visually show the fibrillar assembly atop virus particles. Finally, in vivo testing in a Syrian Hamster model will determine in vivo efficacy of constructs.

Despite broad efficacy in binding several different therapeutic targets, a major gap exists in understanding mechanisms of SAP binding and neutralization of viral particles. SAP demonstrates facile self-assembly into ß-sheets and nanofibers in an aqueous buffer. Supramolecular assembly allows the formation of thixotropic viscous liquids that reversibly shear thin and shear recover—facilitating syringe aspiration and delivery in multiple tissue spaces. Preliminary SPR shows the ability of peptides to specifically bind their intended targets and then further self-assemble atop them. Facile conjugation of spike binding domains (SPd) has preserved self-assembly and conferred an improved IC₅₀ in vitro against live-virus compared to SPd alone. In-house mutation of current SPd domains has resulted in a shorter spike binding peptide with improved affinity (and efficacy) against SARS-CoV-2 and in silico binding to SARS—especially when modified with SAP—termed ESBP3. The present inventors believe that ESBP3 will be efficacious against the B.1.1.7, B.1.351 and P.1 SARS-CoV-2 variants and SARS CoV through a mechanism of epi-viral spike targeting and self-assembly, with demonstrable efficacy in vivo. To date, little is known about how SAP (in particular ESBP3) binds and block viral entry. Preliminary data provide a strong basis to further investigate the mechanism by which SAP demonstrates robust binding, inhibition, and therapeutic effect. Specifically, ESBP3 super stoichiometric binding kinetics, visualization of epi-viral self-assembly and pan-coronavirus in vitro/SARS-CoV-2 in vivo efficacy will be investigated in live-virus models.

Aim 3.1. Efficacy of ESBP3 against novel mutants. ESBP3 will be synthesized (SPPS) and characterized (HPLC-MS) as previously. To determine in vitro efficacy: 4×10⁵ Vero cells/well will be seeded on a 6 well plate using DMEM supplemented with L-glutamine and 10% FBS. At 18-24 hours post-seeding, 30-40 plaque forming units of SARS-CoV-2 in 400 uL was prepared in serum free DMEM and mixed with the ESBP3 (at 0.1, 1, 10, 100, 1000, 10 000 nM) and incubated at 37 C for 1 hr (untreated virus control maintained in each plate). The Vero cell monolayer will be washed with PBS and virus-peptide complex or virus added and incubated at 37° C. for 1 hr. The inoculum will be removed carefully and 4 mL agarose-DMEM overlay added to each well. The plates will be allowed to solidify at room temperature for ˜15 mins and transferred to 37° C. incubator with 5% CO₂. On day 3, the plates will be fixed with 10% buffered formalin for 30 mins and washed with PBS. Agar plugs will be removed and stained with 0.2% crystal violet (in 20% ethanol) for 10 mins. The wells will be washed with water, dried and plaques counted. Similarly, SARS (Urbani Strain) will be obtained and ESBP3 inhibition will be evaluated in a dose-dependent manner. Controls with REGN-CoV2 (Combo of casirivimab/REGN10933 & imdevimab/REGN10987) ab and 1×PBS carrier will be used (n=4 for all assays). After optimization of IC₅₀ as above (prel. 10 nM), in vivo testing in a Syrian Hamster model will determine in vivo efficacy of constructs as we elucidate the mechanism of epiviral self-assembly of ESBP3.

Aim 3.2. Determine a mechanism of action for ESBP3. SPR and cryo-EM will be used to determine affinity and visualize epi-viral binding, coated virus phagocytosis—complemented by a world-leader in these techniques at BCM (MPI Prasad). SPR: His-tagged Spike and novel mutant Spikes will be used as immobilization ligands on an NTA chip, after which ESBP3 will be passed through at the above concentrations.

Super stoichiometric binding will be observed and quantified and compared to the scrambled peptide (ESBP3sc), REGN-CoV2 against Spike, and 1×PBS carrier control. Cryo-EM imaging (Co-I Prasad): live-virus incubated with ESBP3 (fixed and decontaminated) for 1 hr at 10 and 100 nM, will be imaged to note phagocytosis and epi-viral self-assembly. Briefly, live-virus titers (1×10⁷ PFU) will be incubated with 1 uM or 10 uM of ESBP3 for 1 hr. Samples will then be centrifuged at 500×g for 15 min to precipitate fibers and bound viral particles (vs. ultracentrifugation required to precipitate viral particles alone). Samples will be decanted and neutralized with the addition of 2w % glutaraldehyde for 24 hr, centrifuged again, and fixed for another 24 hr in a secondary container. Subsequently, samples will be washed with 1×PBS buffer and prepared for Cryo-EM. Negatively stained TEM will be performed on Vero infected cells (at 24 hr, Aim 1.1 at IC₅₀ concentration, fixed in 2.5% glut as above 2×) and stained to determine internalization of nanofiber coated viral particles, in we have published previously for cells/fibers alone. These studies with neutralized viral titers preincubated with SAP opsonins will mechanistically and visually show the fibrillar assembly atop virus particles.

Aim 3.3. Pre-exposure prophylaxis and post-exposure treatment (virtual mask). Critical to the widespread adoption of ESBP3 as a COVID therapeutic and for the translation of the SAP platform, the use of SAP to coat the nasal passageways preventing infection is paramount. Either pre-exposure (nasal spray in the AM) or post-(potential) exposure (in the PM after work)—will be evaluated for preventing infection and spread. In an established Golden Syrian hamster model—ESBP3 will be dosed at 1 mM, 0.1 mM and 0.01 mM IV or IN and then given an intranasal inoculum of SARS-CoV-2 (at 4 hrs).

Similarly, in another group, after infection, ESBP3 will be delivered after 4 hrs, simulating post-exposure prophylaxis. Hamsters will then be housed in pairs and lung viral load (PCR), lung histopathology delineating the extent of infection and rapidity of viral inhibition measured at 4 days (max viral load) and 7 days (established organ histopathology). (n=8×3(+1 PBS control) concentrations×2 delivery methods×2 exposures×2 timepoints=256 hamsters). Blood will be analyzed for IgG specific to ESBP3 or Spike (by precoating ELISA plates with ESBP3 and Spike overnight at 4° C., washing and probing with plasma and a Dk anti-hamster HRP secondary). Critically—the lack of an immune response to ESBP3 (as seen in Prel. data) and potentially faster development of IgG response to Spike protein may be seen due to viral opsonization and phagocytosis visualized in Aim 2 and preliminary data.

Success will be measured by demonstration of in vitro live-virus efficacy of ESBP3 against mutant strains of SARS-CoV-2, SARS and determination of an IC₅₀ value; determination of the K_{D} (Kon/K_{off}) of ESBP3 and super stoichiometric binding, visualize binding of fibers to virus (cryo-EM) and potential internalization into vacuoles (negatively stained EM); determination of the efficacy of ESBP3 in preventing infection of COVID-19, treat infection and prevention of the communal spread of infection.

Aim 4: Translation—optimizing intranasal delivery, large animal PK and facile SAP GMP manufacturing. Preliminary data shows (10 nM/μg efficacy) 10 mM IV safety through daily dosing and non-GMP gram-scale syntheses. Canine intranasal delivery which are shown in mice herein will be utilized. Homogenization of tissue and determination of mouse/canine intranasal and large animal IV PK and biodistribution will be done as a further objective.

Results show the ability to deliver repeat dose (10 days) ESBP3 intranasally in mice with safety in repeat dose (10 days) IV rat studies. Importantly, the IN and IV doses used in rats were mM, while the IC₅₀ with live-virus was ˜10 nM. Notwithstanding the orders of magnitude of safety over efficacious doses, IN dosing must be optimized and PK evaluated in rodent and canine species. Further canine IV safety must be established as is conventional (2 species toxicology and PK) as part of the IND DMPK package. The novel compositions developed herein showed excellent shelf storage (>3 months @25 C, with <10% lyophilized peptide degradation of SAPs) and indicates the ability of these peptides to remain stable without the need for a dedicated/complicated cold-chain. In this Aim, it was also hypothesized that ESBP3 constructs' safety and PK profile with not change up to mM doses in large animal models. Further, it was hypothesized that facile translatable scale-up of GMP manufacturing from pilot lab-scale (50-100 mg) and CRO non-GMP-scale (500 mg-1 g).

Aim 4.1 Optimizing intranasal delivery in vitro. While intranasal delivery in rodents (1 mM, 15-50 μL) can be delivered using drops of fluid on the nostril, aerosolization of ESBP3 in the nasopharyngeal region is critical to translation. Preliminarily, ESBP3 at 1 mM has shown aerosolization and ˜100% delivery from a syringe using the commercially available Teleflex® Intranasal Mucosal Atomization Device (MAD). Building upon this, ESBP3 delivery at different concentrations (1 mM, 0.1 mM, 0.01 mM and 0.001 mM) will be determined by the collection of atomized sprays in a 50 mL tube and UV spec concentration quantitation versus a standard curve (n=8 per spray concentration).

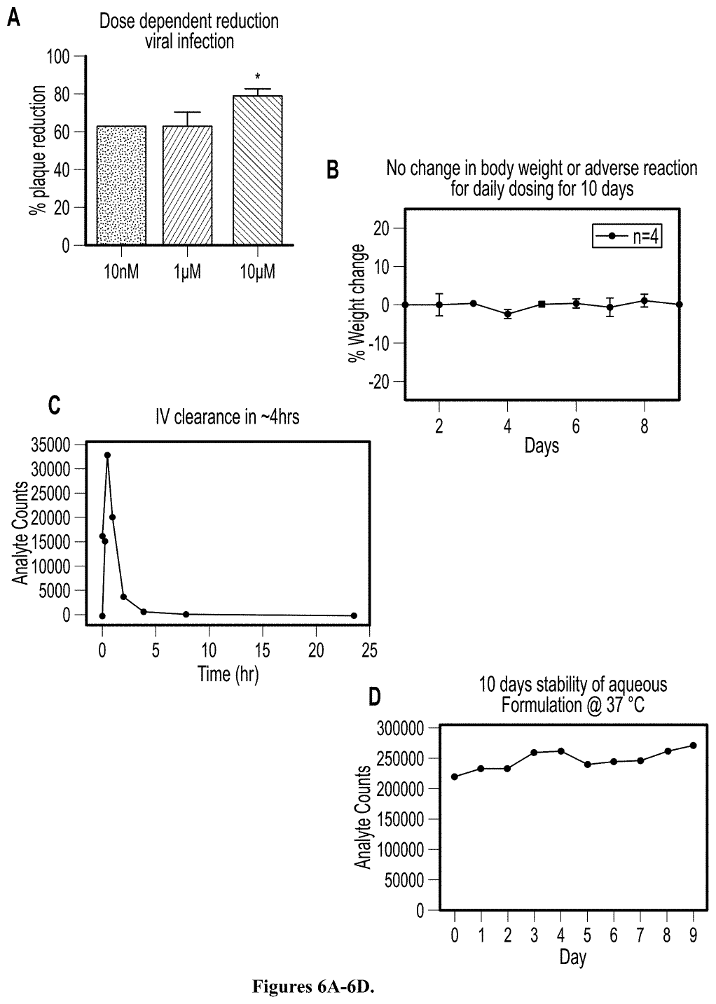

Aim 4.2. Determine IN and IV PK (GLP). ESBP3 shows an excellent safety profile (daily >1 mM @ 1% blood volume—˜200 μL—in rats IV) and clearance within 4-6 hours, FIG. 6A-6D. Rats will be dosed IN (25 μL per nostril at 0.1 and 1 mM) (IV completed) and blood drawn serially (every 10 mins up to 1 hr, and then every 30 mins up to 4 hr, and then at 24 hr daily) to note absorption into the circulation (n=8 for single-dose and 10 day recovery period; and n=8 for repeat daily dose for 10 days and 10 days recovery). Nasal lavage will be performed to quantitate the delivery of peptide daily prior to the next (daily) repeat dose. At 10 days (single dose) and 20 days (repeat dose), sacrifice rats. Canine PK (scaled up to 0.1 and 1 mM at 1 mL/kg) will be used to determine IV clearance after a single dose and repeat dose ESBP3 assayed from an indwelling catheter. Briefly, adult beagles (22-26 lbs) will be enrolled, catheterized and dosed at 0.1 and 1 mM IV or IN (0.5 mL per nostril at 0.1 and 1 mM) ESBP3 daily for 10 days (covering ˜3× the 3-day course of treatment planned in humans; similar to other drugs in Phase I trials); with 10 days recovery (n=4-2 male and 2 female as per ICH standards for each of 2 groups+ carrier control). Blood (plasma) will be collected at every 10 mins to 1 hr, and then every 30 mins to 4 hr, and then at 24 hr daily, assuming a similar PK pattern to rats. Nasal lavage will be collected daily prior to the second dose and during recovery. At 20 days, animals will be sacrificed, and organs harvested for histo-path. Plasma and nasal lavage (and turbinate homogenates from rats) will be frozen and shipped to TDM Pharma (our contract LC/MS provider who has developed a fit for purpose plasma and tissue homogenate LC/MS method in rodents and canines for ESBP3 and other SAP); whole organ histopathology will be performed by the Rutgers Pathology Core as per ICH guidelines to determine and local or systemic toxicity of ESBP3. Plasma from terminal blood draws will also be used in sandwich ELISA (Aim 1.3) to detect the presence of anti-drug antibodies (ADA—none seen to date) of this short peptide in rat and canine species. (48 rats and 24 canines used in total.)

Shown in FIGS. 6A-6D are preliminary safety and efficacy of ESBP2. FIG. 6A illustrates an in vitro dose dependent reduction in live-virus infectivity at <10 nM IC₅₀ along with excellent A549 cell cytocompatibility (data not shown), prompted safety testing in vivo. FIG. 6B illustrates a 10 day repeat dosing in rats at 1% blood volume of 1 mM peptides showed no adverse reaction with FIG. 6C showing an IV clearance in approximately 4 hrs. In FIG. 6D, notably, the benchmarked LC/MS method for plasma and peptide API showed excellent stability of drug formulation at 37° C. for over 10 days.

Aim 4.3. GMP scale-up. Lab-scale 10-100 mg, CMO non-GMP 0.5-1 gram scale syntheses demonstrate the facile ability to synthesize these peptides using conventional SPPS methods. The present inventors are confident in the facile synthesis (adapting identical methods as the non-GMP batch) of cGMP 5 g ESBP3 and a 1 g reference standard batch. From this 5 g GMP batch, performed will be stability testing of the API and formulation (in 0.9% saline) at −20° C. and 5° C. (1 week, 1 month, 3, 6, 9,18, 24 month) at −20° C., 5° C. and accelerated stability at 25° C./65% RH (1, 3 and 6 month) (accelerated stability testing). Success will be measured by optimizing the delivery of peptide with <10% material loss in the mechanical aerosolizer; verifying IV plasma PK in canines compared to rats, optimizing intranasal dosing (in 2 species), demonstrating a biodistribution (IN) and IN PK profile (in 2 species), modulating dosing (if necessary preliminary data has shown 10-day repeat dose of 1-10 mM dosing in mice and rats IN and IV shown to be safe); and the lack of ADA (in 2 species); verifying lab and non-GMP scale syntheses at GMP scale, with proving the stability of lyophilized peptide and formulation. Preliminary data for SAP shows >90% stability over several months at ambient or accelerated degradation conditions.

Aim 5: Platform−computational design of a pan-coronavirus targeting SAP. Preliminary data shows the ability for ESBP3 to target SARS-CoV-2, SARS and other coronaviruses due to its short promiscuous nature but robust efficacy due to epi-viral self-assembly. First, the multivalency afforded by functionalized SAP targeting may explain enhanced SBP3 binding that will be modeled in silico using Rosetta and MD simulations with Col Kral. Next the sequence is mutated for a canonical sequence against SARS-CoV-2 variants: B.1.1.7, B.1.351 and P.1; and MERS, SARS-HCoV, HCoV-229E, HCoV-NL63, HCoV-OC43, and HCoV-HKU1 coronaviruses.

Coupling with SAP: ESBP1 showed improved efficacy compared to published SBP1 sequences. Through site-specific mutation and complimentary docking to SARS as well, designed were the SAP—ESBP3 that has ˜10 nM IC₅₀—3 orders of magnitude improved over published SBP1; with in silico targeting of SARS. Given the rise of mutants, the rational design of SAP-SBP constructs against crystal structures can allow the development of pan-coronavirus therapeutics.

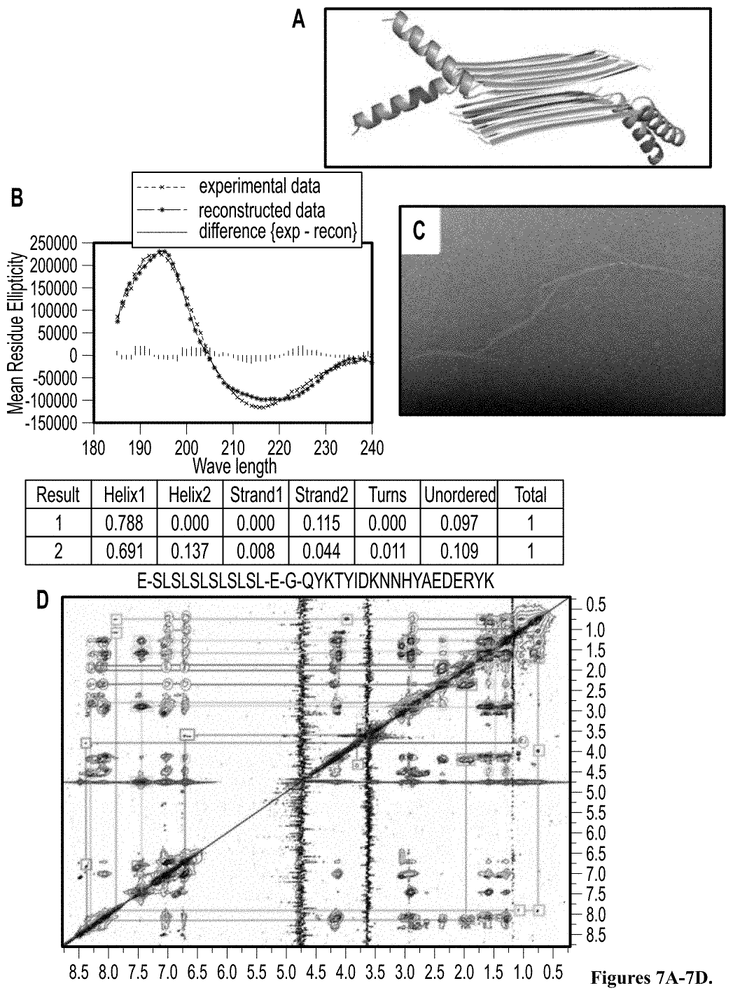

Aim 5.1. Develop a computational model for Spike binding of ESBP3. The multivalency afforded by functionalized SAP targeting may explain enhanced SBP3 binding to Spike protein. Further Spike trimerization (and availability of epi-viral assembled ESBP3 can allow enhanced local binding and blocking of Spike protein—explaining orders of magnitude improved IC₅₀ for ESBP3 compared to SBP3 alone. Ongoing NMR experiments will help determine ESBP-3 peptide-peptide interactions, specifically 1D proton, ¹H, ¹³C-HSQC, 2D ¹H,¹H-NOESY, 2D DOSY, T1/T2 measurement and deuterium exchange as shown in FIGS. 7A-7D. NMR NOE constraints will be incorporated into Rosetta for ESBP3 and SBP3 modeling on Spike protein, complementing TEM and CD reconstruction of the structure as also shown in FIGS. 7A-7D.

FIGS. 7A-7D illustrate structural analysis of ESBP3. FIG. 7A is a schematic of ESBP3 minimized in Rosetta—showing β-sheet self-assembly into fibers and folding of the SBP3 helix. FIG. 7B is a peptide modelling bolstered by circular dichroism showing characteristic β-sheet and α-helix signatures, deconvoluted to give approximate percentage contribution to secondary structure. FIG. 7C is a negatively stained TEM having FIG. 7D showing NMR analysis of ESBP3. An overlay showing the TOCSY spectra in black, and NOESY spectra in red. Chemical shift assignments in the TOCSY are indicated by boxes and adjacent peaks connected through double lines (residue color in the sequence is used to differentiate correlated peaks). NOE cross peaks which can be used to spatially fix the peptide are indicated by circles, and represent a mix of intra- and inter-residue, as well as intra- and inter-chain interactions. Because of the repetitive peptide sequence and symmetry of the nanofiber (composed pairs of oriented dimers translated spatially in one dimension) there are a large number of overlapping and/or identical chemical environments which cannot be unambiguously assigned without the addition of site selective NMR labels.

Building this SBP3-RBD modeling, ESBP3-Spike (±trimerization±self-assembly) will be modeled in silico using Rosetta and MD simulations will be performed by MPI Kral in order to quantify the stability of the nanofiber-Spike complex as well as identify significant interactions that explain the high affinity of ESBP3 versus SBP3. Different self-assembling backbones (ex: KSBP3) will also be analyzed through NMR experiments, Rosetta modeling and NAMD simulations to quantify the impact of peptide design on empirical IC₅₀s. These types of structures will assist in the analysis and modeling SBP3 and ESBP3 applicability for SARS-CoV-2 variants and other coronaviruses.

Aim 5.2. Develop a pan-coronavirus inhibitor. Next, improving the sequence to identify a canonical sequence targeting SARS-CoV-2 variants: B.1.1.7, B.1.351 and P.1; and MERS, SARS-HCoV, HCoV-229E, HCoV-NL63, HCoV-OC43, and HCoV-HKU1 coronaviruses is done. Rosetta Scripts will be implemented for iterative mutations of SBP3 docked with SARS-CoV-2 variants' Spike RBD as crystal structures using the methodologies outlined in FIGS. 5A-5F and 6A-6D. Subsequently, this putative sequence will be used against other coronaviruses based on candidate crystal structures for each aforementioned virus: MERS, SARS-HCoV, HCoV-229E, HCoV-NL63, HCoV-OC43, and HCoV-HKU1. Alterations in the Spike RBD as well as different outputs on Rosetta Scripts/Coupled Moves mutagenesis will dictate significant and insignificant sequence changes for SBP3 binding of each RBD. SBP3 mutants output from Rosetta Scripts will be validated by molecular docking via HADDOCK and NMR investigation to confirm crucial and expected interactions between SBP3 mutants and Spike proteins. Promising peptide candidates will be tested by MD with different viral strain's Spike RBDs to confirm binding stability once the sequence has been modified to contain an SAP. Experimental binding kinetics of SBP3 mutants will be determined through SPR against a wide array of variants and coronaviruses mentioned previously.

Aim 5.3. Modular design of hetero-functional peptide binders. Self-assembly allows the addition of terminally modified monomers with identical self-assembling backbones to co-assemble into hetero-functional supramolecular fibers, for example including but not limited to FIG. 2A with different “bioactive” domains. These assemblies may offer a one-dose treatment for multiple variants, which vaccines and other mAb therapeutics may not affect (paradoxically due to their high specificity). Owing to the shorter peptide domain—with potentially lower Kon, the promotion of super-stoichiometric binding allows for moderately specific self-assembling peptides (IC50˜nM vs pM for Ab) to bind irreversible (K_{D} much greater than mAb). Here, SBP3 mutants will be fabricated along with SBP3, and test their in vitro SPR binding affinity to viral variant Spikes', and then test their combinations (ratios of peptides varying from (1:99, 5:95, 10:90, 25:75 and 50:50) will be assayed for combinatorial efficacy against 2 different live-virus strains that show differing (SBP3 alone) inhibition rates, along with dose-dependence (0.1 nM-100 μM) to discover potential synergies between rationally designed constructs.

Although the invention herein has been described with reference to embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### REFERENCES

1. Zhang, G., Pomplun, S., Loftis, A. R., Loas, A. & Pentelute, B. L. The first-in-class peptide binder to the SARS-CoV-2 spike protein. bioRxiv, 2020.03.19.999318 (2020).
2. Ledford, H. How does COVID-19 kill? Uncertainty is hampering doctors' ability to choose treatments. Nature (2020).
3. Gewin, V. Safely conducting essential research in the face of COVID-19. Nature (2020).
4. Gotian, R. Mentoring during the COVID-19 pandemic. Nature (2020).
5. Jiang, S. Don't rush to deploy COVID-19 vaccines and drugs without sufficient safety guarantees. Nature 579, 321 (2020).
6. Liu, C. et al. Research and Development on Therapeutic Agents and Vaccines for COVID-19 and Related Human Coronavirus Diseases. ACS Cent Sci 6, 315-331 (2020).
7. Shetty, A. K. Mesenchymal Stem Cell Infusion Shows Promise for Combating Coronavirus (COVID-19)-Induced Pneumonia. Aging Dis 11, 462-464 (2020).
8. Aronson, J. K. & Ferner, R. E. Drugs and the renin-angiotensin system in covid-19. BMJ 369, m1313 (2020).
9. Casadevall, A. & Pirofski, L. A. The convalescent sera option for containing COVID-19. J Clin Invest 130, 1545-1548 (2020).
10. Bedford, J. et al. COVID-19: towards controlling of a pandemic. Lancet 395, 1015-1018 (2020).
11. Mehta, P. et al. COVID-19: consider cytokine storm syndromes and immunosuppression. Lancet 395, 1033-1034 (2020).
12. Ritchie, A. I. & Singanayagam, A. Immunosuppression for hyperinflammation in COVID-19: a double-edged sword? Lancet 395, 1111 (2020).
13. FitzGerald, G. A. Misguided drug advice for COVID-19. Science 367, 1434 (2020).
14. Kupferschmidt, K. & Cohen, J. Race to find COVID-19 treatments accelerates. Science 367, 1412-1413 (2020).
15. Li, G. & De Clercq, E. Therapeutic options for the 2019 novel coronavirus (2019-nCoV). Nat Rev Drug Discov 19, 149-150 (2020).
16. Ferner, R. E. & Aronson, J. K. Chloroquine and hydroxychloroquine in covid-19. BMJ 369, m1432 (2020).
17. Cao, B. et al. A Trial of Lopinavir-Ritonavir in Adults Hospitalized with Severe Covid-19. N Engl J Med (2020).
18. Gautret, P. et al. Hydroxychloroquine and azithromycin as a treatment of COVID-19: results of an open-label non-randomized clinical trial. Int J Antimicrob Agents, 105949 (2020).
19. Grein, J. et al. Compassionate Use of Remdesivir for Patients with Severe Covid-19. N Engl J Med (2020).
20. Gordon, C. J., Tchesnokov, E. P., Feng, J. Y., Porter, D. P. & Gotte, M. The antiviral compound remdesivir potently inhibits RNA-dependent RNA polymerase from Middle East respiratory syndrome coronavirus. J Biol Chem 295, 4773-4779 (2020).
21. Martinez, M. A. Compounds with therapeutic potential against novel respiratory 2019 coronavirus. Antimicrob Agents Chemother (2020).
22. Choy, K. T. et al. Remdesivir, lopinavir, emetine, and homoharringtonine inhibit SARS-CoV-2 replication in vitro. Antiviral Res, 104786 (2020).
23. Dong, L., Hu, S. & Gao, J. Discovering drugs to treat coronavirus disease 2019 (COVID-19). Drug Discov Ther 14, 58-60 (2020).
24. Lu, H. Drug treatment options for the 2019-new coronavirus (2019-nCoV). Biosci Trends 14, 69-71 (2020).
25. Wrapp, D. et al. Cryo-EM structure of the 2019-nCoV spike in the prefusion conformation. Science 367, 1260-1263 (2020).
26. Wang, X. et al. SARS-CoV-2 infects T lymphocytes through its spike protein-mediated membrane fusion. Cellular & Molecular Immunology (2020).
27. Xia, S. et al. Inhibition of SARS-CoV-2 (previously 2019-nCoV) infection by a highly potent pan-coronavirus fusion inhibitor targeting its spike protein that harbors a high capacity to mediate membrane fusion. Cell Res 30, 343-355 (2020).
28. Prajapat, M. et al. Drug targets for corona virus: A systematic review. Indian J Pharmacol 52, 56-65 (2020).
29. Zumla, A., Chan, J. F. W., Azhar, E. I., Hui, D. S. C. & Yuen, K.-Y. Coronaviruses drug discovery and therapeutic options. Nature Reviews Drug Discovery 15, 327-347 (2016).
30. Ortega, J. T., Serrano, M. L., Pujol, F. H. & Rangel, H. R. Role of changes in SARS-CoV-2 spike protein in the interaction with the human ACE2 receptor: An in silico analysis. EXCLI J 19, 410-417 (2020).
31. Ou, X. et al. Characterization of spike glycoprotein of SARS-CoV-2 on virus entry and its immune cross-reactivity with SARS-CoV. Nature Communications 11, 1620 (2020).
32. Yi, H. A., Fochtman, B. C., Rizzo, R. C. & Jacobs, A. Inhibition of HIV Entry by Targeting the Envelope Transmembrane Subunit gp41. Curr HIV Res 14, 283-94 (2016).
33. Mohanty, U. & Dixit, N. M. Mechanism-based model of the pharmacokinetics of enfuvirtide, an HIV fusion inhibitor. J Theor Biol 251, 541-51 (2008).
34. Du, Z., Zhu, F., Guo, F., Yang, B. & Wang, T. Detection of antibodies against SARS-CoV-2 in patients with COVID-19. J Med Virol (2020).
35. Shen, C. et al. Treatment of 5 Critically Ill Patients With COVID-19 With Convalescent Plasma. JAMA (2020).
36. Shanmugaraj, B., Siriwattananon, K., Wangkanont, K. & Phoolcharoen, W. Perspectives on monoclonal antibody therapy as potential therapeutic intervention for Coronavirus disease-19 (COVID-19). Asian Pac J Allergy Immunol 38, 10-18 (2020).
37. Chen, L., Xiong, J., Bao, L. & Shi, Y. Convalescent plasma as a potential therapy for COVID-19. Lancet Infect Dis 20, 398-400 (2020).
38. Bloch, E. M. et al. Deployment of convalescent plasma for the prevention and treatment of COVID-19. J Clin Invest (2020).
39. Lau, J. L. & Dunn, M. K. Therapeutic peptides: Historical perspectives, current development trends, and future directions. Bioorg Med Chem 26, 2700-2707 (2018).
40. Petrak, K. et al. Challenges in Translating from Bench to Bed-Side: Pro-Angiogenic Peptides for Ischemia Treatment. Molecules 24 (2019).
41. Zapadka, K. L., Becher, F. J., Gomes Dos Santos, A. L. & Jackson, S. E. Factors affecting the physical stability (aggregation) of peptide therapeutics. Interface Focus 7, 20170030 (2017).
42. Fosgerau, K. & Hoffmann, T. Peptide therapeutics: current status and future directions. Drug Discov Today 20, 122-8 (2015).
43. Drucker, D. J. Advances in oral peptide therapeutics. Nature Reviews Drug Discovery 19, 277-289 (2020).
44. Bottger, R., Hoffmann, R. & Knappe, D. Differential stability of therapeutic peptides with different proteolytic cleavage sites in blood, plasma and serum. PLoS One 12, e0178943 (2017).
45. Di, L. Strategic approaches to optimizing peptide ADME properties. AAPS J 17, 134-43 (2015).
46. Hall, M. P. Biotransformation and in vivo stability of protein biotherapeutics: impact on candidate selection and pharmacokinetic profiling. Drug Metab Dispos 42, 1873-80 (2014).
47. Moore, A. N. & Hartgerink, J. D. Self-Assembling Multidomain Peptide Nanofibers for Delivery of Bioactive Molecules and Tissue Regeneration. Acc Chem Res 50, 714-722 (2017).
48. Kumar, V. A. et al. Treatment of hind limb ischemia using angiogenic peptide nanofibers. Biomaterials 98, 113-9 (2016).
49. Kumar, V. A., Wickremasinghe, N. C., Shi, S. & Hartgerink, J. D. Nanofibrous Snake Venom Hemostat. ACS Biomater Sci Eng 1, 1300-1305 (2015).
50. Kumar, V. A. et al. Drug-triggered and cross-linked self-assembling nanofibrous hydrogels. J Am Chem Soc 137, 4823-30 (2015).
51. Kumar, V. A. et al. Highly angiogenic peptide nanofibers. ACS Nano 9, 860-8 (2015).
52. Yan, R. et al. Structural basis for the recognition of SARS-CoV-2 by full-length human ACE2. Science 367, 1444-1448 (2020).
53. Zhang, L. et al. Crystal structure of SARS-CoV-2 main protease provides a basis for design of improved alpha-ketoamide inhibitors. Science (2020).
54. Yuan, M. et al. A highly conserved cryptic epitope in the receptor-binding domains of SARS-CoV-2 and SARS-CoV. Science (2020).
55. Kumar, V. A. et al. Self-assembling multidomain peptides tailor biological responses through biphasic release. Biomaterials 52, 71-8 (2015).
56. Shi, S. et al. Development of peptide inhibitors of HIV transmission. Bioact Mater 1, 109-121 (2016).
57. Sarkar, B. et al. Angiogenic Self-Assembling Peptide Scaffolds for Functional Tissue Regeneration. Biomacromolecules 19, 3597-3611 (2018).
58. Ma, X. et al. Angiogenic peptide hydrogels for treatment of traumatic brain injury. Bioact Mater 5, 124-132 (2020).
59. Sarkar, B. et al. Implantable anti-angiogenic scaffolds for treatment of neovascular ocular pathologies. Drug Deliv Transl Res (2020).
60. Shi, S. et al. Evolving role of biomaterials in diagnostic and therapeutic radiation oncology. Bioact Mater 5, 233-240 (2020).
61. Kim, K. K., Siddiqui, Z., Patel, M., Sarkar, B. & Kumar, V. A. A self-assembled peptide hydrogel for cytokine sequestration. J Mater Chem B 8, 945-950 (2020).
62. Wickremasinghe, N. C., Kumar, V. A. & Hartgerink, J. D. Two-step self-assembly of liposome-multidomain peptide nanofiber hydrogel for time-controlled release. Biomacromolecules 15, 3587-95 (2014).
63. Wickremasinghe, N. C., Kumar, V. A., Shi, S. & Hartgerink, J. D. Controlled Angiogenesis in Peptide Nanofiber Composite Hydrogels. ACS Biomater Sci Eng 1, 845-854 (2015).
64. Kumar, V. A., Wang, B. K. & Kanahara, S. M. Rational design of fiber forming supramolecular structures. Exp Biol Med (Maywood) 241, 899-908 (2016).
65. Hitscherich, P. et al. Injectable Self-Assembling Peptide Hydrogels for Tissue Writing and Embryonic Stem Cell Culture. J Biomed Nanotechnol 14, 802-807 (2018).
66. Lopez-Silva, T. L. et al. Chemical functionality of multidomain peptide hydrogels governs early host immune response. Biomaterials 231, 119667 (2020).
67. Nguyen, P. K. et al. Self-Assembly of an Antiangiogenic Nanofibrous Peptide Hydrogel. ACS Applied Bio Materials 1, 865-870 (2018).
68. Carrejo, N. C. et al. Multidomain Peptide Hydrogel Accelerates Healing of Full-Thickness Wounds in Diabetic Mice. ACS Biomater Sci Eng 4, 1386-1396 (2018).
69. Moore, A. N. et al. Nanofibrous peptide hydrogel elicits angiogenesis and neurogenesis without drugs, proteins, or cells. Biomaterials 161, 154-163 (2018).
70. Sarkar, B. et al. Membrane-Disrupting Nanofibrous Peptide Hydrogels. ACS Biomat. Sci. Eng. 5, 4657-4670 (2019).
71. Abdulla, A. et al. Project IDentif.AI: Harnessing Artificial Intelligence to Rapidly Optimize Combination Therapy Development for Infectious Disease Intervention. Advanced Therapeutics n/a, 2000034 (2020).
72. Cohen, J. New coronavirus threat galvanizes scientists. Science 367, 492-493 (2020).
73. Johansson, M. A. & Saderi, D. Open peer-review platform for COVID-19 preprints. Nature 579, 29 (2020).
74. South, A. M., Diz, D. & Chappell, M. C. COVID-19, ACE2 and the Cardiovascular Consequences. Am J Physiol Heart Circ Physiol (2020).
75. Hoffmann, M. et al. SARS-CoV-2 Cell Entry Depends on ACE2 and TMPRSS2 and Is Blocked by a Clinically Proven Protease Inhibitor. Cell 181, 271-280 e8 (2020).
76. Zhou, Y. et al. Network-based drug repurposing for novel coronavirus 2019-nCoV/SARS-CoV-2. Cell Discovery 6, 14 (2020).
77. Lee, S. et al. Self-Assembling Peptides and Their Application in the Treatment of Diseases. Int J Mol Sci 20 (2019).
78. Zhou, J., Li, J., Du, X. & Xu, B. Supramolecular biofunctional materials. Biomaterials 129, 1-27 (2017).
79. Habibi, N., Kamaly, N., Memic, A. & Shafiee, H. Self-assembled peptide-based nanostructures: Smart nanomaterials toward targeted drug delivery. Nano Today 11, 41-60 (2016).
80. Webber, M. J., Appel, E. A., Meijer, E. W. & Langer, R. Supramolecular biomaterials. Nat Mater 15, 13-26 (2016).
81. Wang, M. et al. Remdesivir and chloroquine effectively inhibit the recently emerged novel coronavirus (2019-nCoV) in vitro. Cell Res 30, 269-271 (2020).
82. Wong, C. K. et al. Plasma inflammatory cytokines and chemokines in severe acute respiratory syndrome. Clin Exp Immunol 136, 95-103 (2004).
83. Huang, C. et al. Clinical features of patients infected with 2019 novel coronavirus in Wuhan, China. Lancet 395, 497-506 (2020).
84. Liu, B., Li, M., Zhou, Z., Guan, X. & Xiang, Y. Can we use interleukin-6 (IL-6) blockade for coronavirus disease 2019 (COVID-19)-induced cytokine release syndrome (CRS)? J Autoimmun, 102452 (2020).
85. Zhang, C., Wu, Z., Li, J. W., Zhao, H. & Wang, G. Q. The cytokine release syndrome (CRS) of severe COVID-19 and Interleukin-6 receptor (IL-6R) antagonist Tocilizumab may be the key to reduce the mortality. Int J Antimicrob Agents, 105954 (2020).
86. Lu, R. et al. Genomic characterisation and epidemiology of 2019 novel coronavirus: implications for virus origins and receptor binding. Lancet 395, 565-574 (2020).
87. Roberts, A. et al. Severe acute respiratory syndrome coronavirus infection of golden Syrian hamsters. J Virol 79, 503-11 (2005).
88. McAuliffe, J. et al. Replication of SARS coronavirus administered into the respiratory tract of African Green, rhesus and cynomolgus monkeys. Virology 330, 8-15 (2004).
89. Martina, B. E. et al. Virology: SARS virus infection of cats and ferrets. Nature 425, 915 (2003).
90. Shi, J. et al. Susceptibility of ferrets, cats, dogs, and other domesticated animals to SARS-coronavirus 2. Science (2020).
91. Chan, J. F. et al. Simulation of the clinical and pathological manifestations of Coronavirus Disease 2019 (COVID-19) in golden Syrian hamster model: implications for disease pathogenesis and transmissibility. Clin Infect Dis (2020).
92. Zhou, P. et al. A pneumonia outbreak associated with a new coronavirus of probable bat origin. Nature 579, 270-273 (2020).
93. Corman, V. M. et al. Detection of 2019 novel coronavirus (2019-nCoV) by real-time RT-PCR. Euro Surveill 25 (2020).
94. Xiong, Y. et al. Transcriptomic characteristics of bronchoalveolar lavage fluid and peripheral blood mononuclear cells in COVID-19 patients. Emerg Microbes Infect 9, 761-770 (2020).
95. Han, Y. & Kral, P. Computational Design of ACE2-Based Peptide Inhibitors of SARS-CoV-2. ACS Nano (2020).
96. Nguyen, P. K. et al. Self-Assembly of a Dentinogenic Peptide Hydrogel. ACS Omega 3, 5980-5987 (2018).

What is claimed is:

1. A method of binding a designer peptide unto a foreign sequence, comprising,
conjugating a targeting sequence to a self-assembling peptide (SAP) having a fibrilizing domain for producing a designer peptide to bind a foreign sequence;
self-assembling on the foreign sequence;
preventing binding of the foreign sequence due to stearic hindrance or lack of binding site availability by the designer peptide; and
targeting for destruction the foreign sequence via phagocytosis, wherein the designer peptide functions as an opsonin.

2. The method of claim 1 wherein the targeting sequence includes a short antigenic sequence of 1-200 amino acids in length from [SEQ.2]-[SEQ.3], [SEQ. 13]-[SEQ. 27], [SEQ. 29], or [SEQ.32].

3. The method of claim 1, wherein the foreign sequence is a strain that causes COVID-19, and wherein the fibrilizing domain includes amino acids from [SEQ. 1], [SEQ.4]-[SEQ. 7], or [SEQ. 12].

4. The method of claim 1, wherein the designer peptide is formed from amino acids selected from a group consisting of [SEQ.8]-[SEQ. 11], [SEQ. 28], [SEQ.30], [SEQ.31][SEQ.33], and [SEQ.34].

5. The method of claim 1, wherein the foreign sequence is SARS-CoV-2-RBD.

6. A composition for a designer peptide opsonin against a foreign sequence, comprising:
a fibrilizing domain for forming a self-assembling peptide;
a spacer domain attached to the fibrilizing domain; and
a targeting sequence that is attached to the spacer domain and binds with a foreign sequence.

7. The composition of claim 6, wherein the peptide binds to the foreign sequence that is an antigen and the peptide prevents binding of the foreign sequence due to stearic hindrance and/or lack of binding site availability to a canonical target.

8. The composition of claim 6, wherein the self-assembling peptide further includes a recognition domain that binds to an antigenic sequence, and subsequently self-assembles atop the antigenic sequence, and wherein the antigenic sequence is a part of a viral protein coat.

9. The composition of claim 8, wherein the viral protein coat is part of a Spike protein of SARS-CoV-2.

10. The composition of claim 6, wherein the self-assembling peptide assembles to form a plurality of nanofibers after binding to a target antigen, and wherein the nanofibers are a plurality of peptide oligomers targeted for destruction as foreign sequence via phagocytosis like an opsonin.

11. The composition of claim 6, wherein the self-assembling peptide assembles to form nanofibers including a sequence of Aₘ-(BC)ₙ-Aₘ [SEQ.1];
wherein A is a charged amino acid selected from a group consisting of K, D, E, R, and any combination thereof; and wherein B is either hydrophilic and hydrophobic and C is alternatively hydrophobic or hydrophilic; and
wherein m and n represent repeats, and m is 1-6 and n is 2-20.

12. The composition of claim 6, wherein the spacer domain is a single glycine spacer, or a longer glycine spacer of 1 to 5 glycine.

13. The composition of claim 6, wherein the targeting sequences are N-terminally acetylated and C-terminally amidated.

14. The composition of claim 13, wherein the sequence includes SBP1[SEQ. 2] that binds SARS-CoV-2-RBD.

15. A composition for a designer peptide opsonin against a foreign sequence, comprising:
a fibrilizing domain [SEQ.1]-[SEQ. 7], or [SEQ. 12] for forming a self-assembling peptide (SAP);
a glycol spacer domain attached to the fibrilizing domain; and
a targeting sequence selected from a group consisting of [SEQ. 2]-[SEQ. 3], [SEQ. 13]-[SEQ. 27], and [SEQ. 29]-[SEQ.32] that is attached to the glycol spacer domain and the fibrilizing domain to form a designer peptide opsonin that binds with a foreign sequence.

16. The composition of claim 15, wherein the self-assembling peptide self-assembles atop a COVID-19 pathogen for immune destruction and the self-assembling peptide is a functional COVID-19 opsonin.

17. The composition of claim 16, wherein the self-assembling peptide is intrinsically biodegradable and endocytosed.

18. The composition of claim 17, wherein the self-assembling peptide is composed of L-amino acids that are highly biocompatible sub-Q, IM and IV with no adverse reaction for 10% blood volume replacement with 1w % of the self-assembling peptide as shown in experimental mice or rats.

19. The composition of claim 15, wherein the targeting sequence is [SEQ.2] and the formed, self-assembling peptide is K(SL)₆K-G-IEEQAKTFLDKFNHEAEDLFYQS.

20. The composition of claim 15, wherein the designer peptide opsonin is defined as [SEQ.12] combined with one sequence from the group [SEQ. 16]-[SEQ. 26].

21. A method of binding a pan-coronavirus peptide unto a foreign sequence, comprising,
conjugating a targeting domain to a self-assembling peptide (SAP) having a fibrilizing domain for producing a pan-coronavirus peptide to bind a foreign sequence, wherein the fibrilizing domain includes amino acids from [SEQ. 1], [SEQ.4]-[SEQ. 7], or [SEQ. 12];
self-assembling the pan-coronavirus peptide on the foreign sequence;
preventing binding of the foreign sequence due to stearic hindrance or lack of binding site availability by the pan-coronavirus peptide; and
targeting for destruction the foreign sequence via phagocytosis, wherein the pan-coronavirus peptide functions as an opsonin.

22. The method of claim 21, wherein the pan-coronavirus peptide is defined as a sequence selected from the group consisting of [SEQ. 28], [SEQ. 30], [SEQ. 31], [SEQ. 33], and [SEQ. 34].

23. The method of claim 21, wherein the targeting domain is selected from a group consisting of [SEQ.27], [SEQ. 29], and [SEQ. 32].

24. The method of claim 21 further includes binding the self-assembling peptide (SAP) to a SARS-CoV-2-ACE2 receptor binding domain of spike protein to prevent infection, and self-assembling the SAP atop a virion to promote opsonization.

25. The method of claim 21 further includes facile tuning of the peptide sequence through rationalized computational design for pan-coronavirus targeting of variants.
